(19) United States
(12) Patent Application Publication
(10) Pub. No.: US 2002/0094316 A1
(43) Pub. Date: Jul. 18, 2002

(54) Polypodal chelants for metallopharmaceuticals

(76) Inventor: Shuang Liu, Chelmsford, MA (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 10/033,769

(22) Filed: Dec. 27, 2001

Related U.S. Application Data
(60) Provisional application No. 60/260,618, filed on Jan. 9, 2001.

(51) Int. Cl. A61K 51/00; A61M 36/14

(52) U.S. Cl. 424/1.11

(57) ABSTRACT

Polypodal chelants are disclosed, as well as chelates of the chelants with metal ions to form radiopharmaceutical and radioactive, MRI and X-ray or CT imaging compounds and compositions. Therapeutic and imaging methods of use are also disclosed.

### FIELD OF THE INVENTION

This invention relates to polypodal chelants and the metal complexes thereof, methods of preparing the chelants and metal complexes, and pharmaceutical compositions comprising the polypodal chelants and metal complexes. This invention relates particularly to the use of the new metal complexes as contrast agents in X-ray, CT, MRI imaging, and as radiopharmaceuticals for the diagnosis of cardiovascular disorders such as thromboembolic disease or arteriosclerosis, infectious disease and cancer. This invention also relates to new bifunctional chelants (BFCs) for attaching diagnostic and therapeutic isotopes to target specific biomolecules such as proteins, peptides, peptidomimetics, and non-peptide receptor ligands. In addition, the polypodal chelants are also useful for metal detoxification or the therapeutic delivery of radioisotopes.

### BACKGROUND OF THE INVENTION

Medical imaging modalities, such as MRI, X-ray, gamma scintigraphy, positron-emission tomography (PET) and CT scanning, have become extremely important tools in the diagnosis and treatment of various diseases and illness. Imaging of internal body parts relies on the contrast between the targeted organ and the surrounding tissues. The targeted organs or tissues are visible by the use of a particular metallopharmaceutical contrast agent. In X-ray and CT diagnostics, increased contrast of internal organs, such as the kidney, the urinary tract, the digestive tract, cardiovascular system, tumors, and so forth is obtained by administering a contrast agent that is substantially radio-opaque. In conventional proton MRI diagnostics, the increased contrast of internal organs and tissues may be obtained by administrating compositions containing paramagnetic metal species, which increase the relaxivity of surrounding water protons. In ultrasound diagnostics, improved contrast is obtained by administering compositions having acoustic impedances different from that of blood and other tissues. In gamma scintigraphy, contrast of an internal organ is obtained by the specific localization of a gamma ray emitting radiopharmaceutical.

Attachment of metal ions to biomolecules (BM) such as antibodies, antibody fragments, peptides, peptidomimetics, and non-peptide receptor ligands leads to useful target-specific diagnostic and therapeutic metallopharmaceuticals. These include fluorescent, radioactive and paramagnetic metal ions attached to proteins that can be used as probes in vivo in biological systems and in vitro in analytical systems as radioimmunoassays. For example, attachment of radionuclides to monoclonal antibodies that recognize tumor-associated antigens provides radioimmunoconjugates useful for cancer diagnosis and therapy. The monoclonal antibodies are used as carriers of desired radioisotopes to the tumor in vivo.

Radiopharmaceuticals can be classified into two primary classes: those whose biodistribution is determined exclusively by their chemical and physical properties; and those whose ultimate distribution is determined by receptor binding or other biological interactions. The latter class is often called target-specific radiopharmaceuticals. In general, a target specific radiopharmaceutical can be divided into four parts: a targeting molecule, a linker, a BFC, and a radionuclide. The targeting molecule serves as a vehicle, which carries the radionuclide to the receptor site at the diseased tissue or organ. The targeting molecules can be macromolecules such as antibodies; they can also be small biomolecules: peptides, peptidomimetics, and non-peptide receptor ligands. The choice of biomolecule depends upon the targeted disease or disease state. The radionuclide is the radiation source. The selection of radionuclide depends on the intended medical use (diagnostic or therapeutic) of the radiopharmaceutical. Between the targeting molecule and the radionuclide is the BFC, which binds strongly to the metal ion and is covalently attached to the targeting molecule either directly or through a linker. Selection of a BFC is largely determined by the nature and oxidation state of the metallic radionuclide. The linker can be a simple hydrocarbon chain or a long poly(ethylene glycol) (PEG), which is often used for modification of pharmacokinetics. Sometimes, an anionic poly (amino acid) is used to increase the blood clearance and to reduce the background activity, thereby improving the target-to-background ratio.

The use of metallic radionuclides offers many opportunities for designing new radiopharmaceuticals by modifying the coordination environment around the metal with a variety of chelants. The coordination chemistry of the metallic radionuclide will determine the geometry and solution stability of the metal chelate. Different metallic radionuclides have different coordination chemistries, and require BFCs with different donor atoms and ligand frameworks. For “metal essential” radiopharmaceuticals, the biodistribution is exclusively determined by the chemical and physical properties of the metal chelate. For target-specific radiopharmaceuticals, however, the “metal label” is not totally innocent because the target uptake and biodistribution will be affected by not only the targeting biomolecule but also the metal chelate and the linker. This is especially true for radiopharmaceuticals based on small molecules such as peptides due to the fact that in many cases the metal chelate contributes greatly to the overall size and molecular weight. Therefore, the design and selection of the BFC is very important for the development of a new radiopharmaceutical.

The same principle used for target-specific metallo-radiopharmaceuticals also applies to target-specific MRI contrast and ultrasound agents. Unlike the target-specific metalloradiopharmaceutical, where the excess unlabeled biomolecule can compete with the radiolabeled BFC-BM conjugate and block the docking of the radiolabeled receptor ligand, MRI and ultrasound contrast agents contain no excess unlabeled BFC-BM conjugate. Saturation of the receptor sites will maximize the contrast between the diseased tissues and normal tissue provided that the use of a relatively large amount of metal-BFC-BM chelate does not cause unwanted side effects.

For a therapeutic radiopharmaceutical or an MRI contrast agent, it is especially important to keep the metal chelate intact under physiological conditions, particularly in the presence of native chelators, such as transferrin, which have very high affinity for trivalent lanthanide metal ions. This requires the chelant to form metal chelates with high thermodynamic stability and kinetic inertness.

Several BFC systems such as ethylenediaminetetraacetic acid (EDTA) and diethylenetriaminepentaacetic acid (DTPA), as well as their derivatives, have been reported to form thermodynamically stable metal chelates. EDTA-based BFCs were first developed by Sunberg et al (Nature 1974, 250, 587) in the 1970's. Krejcarek and Tucker (Biochem. Biophys. Res. Commun. 1976, 77, 581) developed an activated DTPA analog via a mixed anhydride, which can be linked to proteins. Later, Hnatowich et al (Science 1983, 220, 613) used the cyclic anhydride of DTPA for the same purpose. These linear BFCs bond to a variety of metal ions like ¹¹¹In or ⁹⁰Y and form thermodynamically stable metal chelates. However, metal chelates of linear BFCs are kinetically labile, which contributes to the loss of radionuclide from the metal chelate and often leads to severe bone marrow toxicity. Gansow et al (Bioconjugate Chem. 1991, 2, 187; Inorg. Chem. 1986, 25, 2772) prepared a series of substituted DTPA analogs, which form metal chelates with improved solution stability.

Polyaza macrocycles have been widely used as chelants for a variety of transition metals. The macrocyclic polyaminocarboxylates such as 1,4,7,10-tetraazacyclo-dodecane-1,4,7,10-tetracetic acid (DOTA) and 1,4,8,11-tetraazacyclo-tetradecane-1,4,8,11-tetracetic acid (TETA) are known to form highly stable metal chelates due to their highly preorganized macrocyclic ligand framework. Their Gd chelates have been widely used as MRI contrast agents. Examples include gadolinium complexes Gd-DOTA (Dotarem™, Guerbet/France), Gd-HP-DO3A (ProHance™, Bracco/Italy), and Gd-DO3A-butrol (Gadovist™, Schering/Germany).

Macrocyclic chelants such as DOTA have also been used as BFCs for the radiolabeling of proteins (antibodies or antibody fragments) and peptides with various diagnostic and therapeutic radionuclides (such as ¹¹¹In and ⁹⁰Y). Meares and coworkers were the first to synthesize macrocyclic BFCs (Anal. Biochem. 1985, 148, 249; Nucl. Med. Biol. 1986, 13, 363; Inorg. Chem . 1987, 26, 3458), which form ⁶⁷Cu and ⁹⁰Y chelates with high thermodynamic stability and kinetic inertness. Macrocyclic chelants with three-dimensional cavities are of particular interest because of the high stability of the metal chelates, the substantial selectivity for certain metal ions, either by enforcing a specific spatial arrangement of donor atoms or by introducing different donor atoms into the ligand backbone, and their capability to adopt a preorganized conformation in the unchelated form. The higher the degree of preorganization of an unchelated ligand, the more stable the complex is.

Preorganization minimizes the freedom of motion of the donor atoms and the chelant framework during the complexation process in such a way that the free ligand has a conformation more similar to that in the complex. Because of the restricted freedom of motion, the loss of entropy in forming the complex is much less, which leads to the increased thermodynamic stability of the metal chelate. Although preorganization is a concept usually applied to macrocyclic and cryptate metal complexes, it is also of some importance for open-chain chelants. For example, metal complexes of CDTA (trans-cyclohexane-diaminetetraacetic acid) are often 2-3 orders of magnitude more stable than those of EDTA (ethylenediamine-tetraacetic acid) because of the restricted motion of the iminodiacetic chelating arms in CDTA.

Preorganization of a polydentate chelator results in not only high thermodynamic stability but also increased kinetic inertness of its metal chelate. This has been exemplified by the fact that the half-life for Gd(DOTA)⁻ in 0.1 M HCl is 60.2 h and 2000 years at pH=6.0 while the complex Gd(DTPA)²⁻ having comparable thermodynamic stability decomposes rapidly under acidic conditions (K_{obs}=1.2×10⁻³ so⁻¹; t_{½}˜1 min). The highly preorganized macrocyclic framework of DOTA forces four aminoacetate chelating arms to adopt a conformation that the metal ion can be wrapped in an N₄O₄ donor set. At the same, it is more difficult for the coordinated acetate to be dissociated from the metal center. Therefore, preorganization should be an important factor in the design of new BFCs for the radiolabeling of biomolecules.

There are several ways to achieve a high degree of preorganization for a polydentate chelant. These include the use of a macrocyclic ligand framework, the use of hydrogen bond(s) to enforce a three dimensional cavity for metal coordination, and the choice of chelating arms. Poly aminocarboxylate ligands based on cyclen are known to be well preorganized and form highly stable lanthanide complexes due to the endocyclic orientation of the nitrogen donors. The siderophore enterobactin forms much more stable Fe³⁺ complex than MECAM does because of the cyclic triester framework and hydrogen bonding (Garrett, T. M., et al. J. Am. Chem. Soc. 1991, 113, 2965-2977; Stack, T. D. P., et al. J. Am. Chem. Soc. 1992, 114, 1512-1514; Tor, Y., et al. J. Am. Chem. Soc. 1992, 114, 6661-6671; Karpishin, T. B., et al J. Am. Chem. Soc. 1993, 115, 182-192; Karpishin, T. B., et al. J. Am. Chem. Soc. 1993, 115, 6115-6125; Meyer, M., et al. J. Am. Chem. Soc. 1997, 119, 10093-10103.). Tripodal peptides with chiral conformations were found to be stabilized by interstrand hydrogen bonds (Yakirevitch, P., et al. Inorg. Chem. 1993, 32, 1779-1787; Dayan, I., et al. Inorg. Chem. 1993, 32, 1467-1475; Tor, Y., et al. J. Am. Chem. Soc. 1992, 114, 6653-6661.). It was also found that hydrogen bonding plays a significant role in the conformation of the uncoordinated tripodal aminephenol ligands (Caravan, P., et al. J. Am. Chem. Soc. 1995, 117, 11230-11238; Yang, L -W., et al. Inorg. Chem. 1995, 34, 4921-4925; Liu, S., et al. Inorg. Chem. 1993, 32, 4268; Liu, S., et al. Inorg. Chem. 1993, 32, 2773; Liu, S., et al. Inorg. Chem. 1993, 32, 1756; Liu, S., et al. Inorg. Chem. 1992, 31, 5400; Liu, S., et al. J. Am. Chem. Soc. 1992, 114, 6081.) The use of a carbon atom as the bridgehead instead of a tertiary nitrogen atom also limits the motion of the aminephenol chelating arms. Renaud, et al (Chem. Commum. 1999, 457-458) reported C₃ symmetrical lanthanide podates organized by intramolecular trifurcated hydrogen bonds.

### SUMMARY OF THE INVENTION

One object of the present invention is to provide polypodal chelants that can rapidly form highly stable metal chelates useful as diagnostic or therapeutic metalloradiopharmaceuticals, or magnetic resonance imaging contrast agents, or X-ray or CT contrast agents.

Another object of the present invention is to provide polypodal chelants, in which the primary amine-nitrogen atoms in the chelant framework are not part of metal chelation. They serve as hydrogen bonding units to keep polypodal strands in the right conformation for metal chelation. The chelating groups are not just limited to carboxylates and may contain groups such as phosphonate, phosphinate, hydroxamate, hydroxylethyl, and hydroxyaryl. The carboxylic acid functionality can be used for attachment of biomolecules such as proteins and peptides. Thus, the polypodal chelants can also serve as bifunctional chelators (BFCs) for attaching metal ions to bio-directing groups, including proteins, peptides, peptidomimetics, and non-peptides that bind in vivo to a receptor or enzyme that is expressed or up-regulated at a site or in a disease state. The target specific metallopharmaceuticals of the present invention are useful in the diagnosis of disease by magnetic resonance imaging, PET imaging, gamma scintigraphy or in the treatment of disease by systemic radiotherapy.

Another object of the present invention is to provide polypodal chelants, in which the nitrogen heteroatoms incorporated into a preorganized macrocyclic backbone are used only as bridging atoms to connect the chelating arms with the cyclic chelant framework. The chelating groups are not just limited to carboxylates and may contain groups such as phosphonate, phosphinate, hydroxamate, hydroxylethyl, and hydroxyaryl. These chelants may serve as BFCs for attaching metal ions to bio-directing groups including proteins, peptides, peptidomimetics, and non-peptide receptor ligands that bind in vivo to a receptor or enzyme that is expressed or up-regulated at a site or in a disease state. The target specific metallopharmaceuticals of the present invention are useful in the diagnosis of disease by magnetic resonance imaging, PET imaging, gamma scintigraphy or in the treatment of disease by systemic radiotherapy.

Another object of the present invention is to provide polypodal chelants, in which the linker groups connecting the chelating arms and the preorganized chelant framework are alkylene, alkylenecarbonyl, alkylene(hydroxyphosphoryl), alkylenesulfonyl functionalities. The carbonyl, hydroxyphosphoryl and sulfonyl groups bond strongly to either primary amine groups of a tripodal polyamine or secondary amine-nitrogen atoms of a polyaza macrocycle. The other end of each alkylene group is attached to a chelating arm so that three or four chelating arms can form a “crab-like” structure for metal chelation.

Another object of the present invention is to provide polypodal chelants, in which a macrocyclic peptide backbone is used to provide a 3-dimensional ligand framework. In this way, the chelating arms are preorganized for metal chelation. Macrocyclic peptides are of particular interest due to the specific stereochemistry of naturally occurring amino acids (such as aspartic acid, glutamic acid, lysine, and 2,3-diaminopropionic acid), and well-established synthetic methods, particularly the use of peptide synthesizer.

The utility of these new polypodal chelants and their metal chelates depends on the choice of chelating arms. For example, if chelating groups are all phosphonomethyl (CH₂PO₃H₂) or a combination of carboxymethyl (CH₂COOH) and phosphonomethyl groups, the radiolanthanide complexes can be used as therapeutic radiopharmaceuticals for bone-pain palliation or bone marrow metastases. The utility of these polypodal chelants also includes being used as chelators for the treatment of heavy metal intoxication, and as therapeutic agents themselves for the treatment of metabolic bone diseases such as osteoporosis if chelating groups are all phosphonomethyl (CH₂PO₃H₂) and labeled with ^{32/33}P since these polyphosphonate ligands have high binding affinity towards the bone.

The present invention thus relates to polypodal chelants and metal complexes thereof, methods of preparing the chelants and metal complexes, and pharmaceutical compositions comprising the polypodal chelants and metal chelates. This invention relates particularly to the use of new metal chelates of as contrast agents in X-ray, CT, MRI imaging, and radiopharmaceuticals for the diagnosis of cardiovascular disorders such as thromboembolic disease or arteriosclerosis, infectious disease and cancer. This invention also relates to new bifunctional chelants (BFCs) for attaching diagnostic and therapeutic isotopes to target specific biomolecules such as proteins, peptides, peptidomimetics, and non-peptide receptor ligands binding in vivo to a receptor or enzyme that is expressed or up-regulated at a site or in a disease state. The target specific metallopharmaceuticals of the present invention are useful in the diagnosis of disease by magnetic resonance imaging X-ray, CT, PET or scintigraphy or in the treatment of disease by systemic radiotherapy. In addition, the polypodal chelants are also well suited for metal detoxification or therapeutic delivery of radioisotopes.

According to one embodiment of the present invention, a polypodal chelant is provided, having the formula:

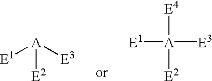

and pharmaceutically acceptable salts thereof, wherein

A is a spacer selected from: R¹—C, R¹—Si, R¹—Ge, N, P, P(O), or a macrocyclic group having the formula:

—[C(L)R²(CR³R⁴)ₐ]_{b}—,

—[N(L)C(W)(CR⁵R⁶)_{c}]_{d}—,

—[OC(W)C(L)R⁷(CR⁸R⁹)ₑ]_{f}—

or

—{[NR¹⁰C(W)C(L)R¹¹(CR¹²R¹³)_{g}]ₕ[NR¹⁴C(W)(CR¹⁵R¹⁶)ᵢ]ⱼ}—,

wherein

a is an integer selected from: 1 to 3;

b is an integer selected from: 3 to 5;

c is an integer selected from: 1 to 3;

d is an integer selected from: 3 or 4;

e is an integer selected from: 1 to 3;

f is an integer selected from: 3 or 4;

g is an integer selected from: 1 to 3;

h is an integer selected from: 3 or 4;

i is an integer selected from: 1 to 3;

j is an integer selected from: 0 to 3;

L is a direct bond to E¹, E², E³, and E⁴;

W is selected from H₂ and O;

R¹, R², R³, R⁴, R⁵, R⁶, R⁷, R⁸, R⁹, R¹⁰, R¹¹, R¹², R¹³, R¹⁴, R¹⁵, and R¹⁶ are independently selected at each occurrence from: H, C₁-C₆ alkyl, C₃-C₆ cycloalkyl, C₁-C₆ fluoroalkyl, C₁-C₆ alkenyl, C₃-C₆ cycloalkenyl, C₁-C₆ fluoroalkenyl, benzyl, fluorobenzyl, phenyl and fluorophenyl;

E¹, E², E³, and E⁴ are chelating arms independently selected from groups having the formula:

(CR¹⁷R¹⁸)ₖ—Z—X—(CR¹⁹R²⁰)ₗNR²¹R²²

wherein

k is an integer selected from 0 to 3, provided that when A is N or —[N(L)C(W)(CR⁵R⁶)_{c}]_{d}—, k is 1-3;

1 is an integer selected from 1 to 3;

X is selected from a group: C(O), S(O)₂, or P(O)(OR¹);

Z is selected from a group: O, NH, NR¹NR¹, ONH or N(OR¹);

R¹⁷, R¹⁸, R¹⁹, R²⁰, R²¹ and R²² are independently selected from: H, C₁-C₁₀ alkyl substituted with 0-5 R²³, C₁-C₁₀ fluoroalkyl substituted with 0-5 R²³, C₂-C₁₀ alkenyl substituted with 0-5 R²³, C₂-C₁₀ fluoroalkenyl substituted with 0-5 R²³, aryl substituted with 0-5 R²³, and fluoroaryl substituted with 0-5 R²³; or R¹⁷ and R¹⁸, or R¹⁹ and R²⁰, or R²¹ and R²² may be taken together to form a C₃-C₁₀ cycloalkyl or C₃-C₁₀ cycloalkenyl optionally interrupted with C(O)NH, NH, NHC(O), NHC(O)NH, NHC(S)NH, O, S, S(O), S(O)₂, P(O)(OR²⁴), P(O)(OR²⁴)O or P(O)(NHR²⁴)O, or to form a ═CH—R^{22a} group, wherein ^{22a} R is aryl substituted with 0-5 R²³, or heterocycle substituted by 0-5 R²³;

R²³ is selected from: H, OH, C(═O)R²⁴, C(═O)OR²⁴, C(═O)NR²⁴₂, PO(OR²⁴)₂ and S(O)₂OR²⁴;

R²⁴ is selected from: H, C₁-C₆ alkyl, C₃-C₆ cycloalkyl, C₁-C₆ fluoroalkyl, C₁-C₆ alkenyl, C₃-C₆ cycloalkyl, C₁-C₆ fluoroalkenyl, benzyl, fluorobenzyl, phenyl and fluorophenyl.

According to another embodiment of the present invention, a radiopharmaceutical compound is provided, in which the polypodal chelant of the present invention is chelated with a radionuclide selected from: ⁶⁰Cu, ⁶²Cu, ⁶⁴Cu, ⁶⁷Cu, ⁶⁷Ga, ⁶⁸Ga, ^{99m}Tc, ¹¹¹In, ⁹⁰Y, ¹⁴⁹Pr, ¹⁵³Sm, ¹⁵⁹Gd, ¹⁶⁶Ho, ¹⁶⁹Yb, ¹⁷⁷Lu, ¹⁸⁶Re and ¹⁸⁸Re.

According to another embodiment of the present invention, an MRI contrast agent is provided in which the polypodal chelant of the present invention is chelated with a paramagnetic metal ion of atomic number 21-29, 42-44, or 58-70.

According to another embodiment of the present invention, an X-ray or CT contrast agent is provided, in which the polypodal chelant of the present invention is chelated with a heavy metal ion of atomic number 21-31, 39-49, 50, 56-80, 82, 83, 90.

According to yet another embodiment of the present invention a conjugate is provided having the formula:

BFC—Lₙ—BM,

and pharmaceutically acceptable salts thereof,

wherein BFC is the bifunctional polypodal chelant of the present invention in which one of R¹ to R²⁴ includes a bond to Lₙ;

Lₙ is a linking group having the formula:

L¹—[Y¹(CR²⁵R²⁶)_{f}(Z¹)_{f″}Y²]_{f′}—L²,

wherein

L¹ is —[(CH₂)_{g}Z¹]_{g′}—(CR²⁵R²⁶)_{g″}—;

L² is —(CR²⁵R²⁶)_{g″}—[Z¹(CH₂)_{g}]_{g′}—;

g is independently 0-10;

g′ is independently 0-1;

g″ is independently 0-10;

f is independently 0-10;

f′ is independently 0-10;

f″ is independently 0-1;

Y¹ and Y², at each occurrence, are independently selected from: a bond, O, NR²⁶, C═O, C(═O)O, OC(═O)O, C(═O)NH—, C═NR²⁶, S, S(O), S(O)₂, NHC(═O), (NH)₂C(═O) and (NH)₂C═S;

R²⁵ and R²⁶ are independently selected at each occurrence from: H, C₁-C₁₀ alkyl substituted with 0-5 R²⁷ and alkaryl wherein the aryl is substituted with 0-5 R²⁷ ;

R²⁷ is independently selected at each occurrence from: NHR²⁸, C(═O)R²⁸, OC(═O)R²⁸, OC(═O)OR²⁸, C(═O)OR²⁸, C(═O)NR₂²⁸, CN, SR²⁸, S(O)R²⁸, S(O)₂R²⁸, NHC(═O)R²⁸, NHC(═O)NHR²⁸, NHC(═S)NHR²⁸ and a bond to BM;

R²⁸ is independently selected at each occurrence from: H, C₁-C₆ alkyl, benzyl, phenyl and a bond to BM; and

BM is a biologically active molecule selected from: IIb/IIIa receptor ligands, fibrin binding peptides, leukocyte binding peptides, chemotactic peptides, LTB₄ receptor antagonists, somatostatin analogs, selectin binding peptides, vitronectin receptor antagonists, and tyrosine kinase inhibitors, matrix metalloproteinase inhibitors, oligonucleotides, fatty acids, nitroimidazoles and carbohydrates.

According to another embodiment of the present invention a radiopharmaceutical conjugate is provided, in which the polypodal chelant of the conjugate of the present invention is chelated with a radionuclide selected from: ⁶⁰Cu, ⁶²Cu, ⁶⁴Cu, ⁶⁷Cu, ⁶⁷Ga, ⁶⁸Ga, ^{99m}Tc, ¹¹¹In, ⁹⁰Y, ¹⁴⁹Pr, ¹⁵³Sm, ¹⁵⁹Gd, ¹⁶⁶Ho, ¹⁶⁹Yb, ¹⁷⁷Lu, ¹⁸⁶Re, and ¹⁸⁸Re.

According to another embodiment of the present invention, an MRI contrast agent is provided, in which the polypodal chelant of the conjugate of the present invention is chelated with a paramagnetic metal ion of atomic number 21-29, 42-44 or 58-70.

According to another embodiment of the present invention, an X-ray or CT contrast agent is provided, in which the polypodal chelant of the conjugate of the present invention is chelated with a heavy metal ion of atomic number 21-31, 39-49, 50, 56-80, 82, 83 or 90.

According to another embodiment of the present invention, intermediate compounds are provided having the formula:

A[(CR¹⁷R¹⁸)ₖNH₂]ₘ

wherein A is a spacer selected from R¹—C, R¹—Si, R¹—Ge, N, P and P(O), or a macrocyclic group having the formula:

—[C(L)R²(CR³R⁴)ₐ]_{b}—,

—[N(L)C(W)(CR⁵R⁶)_{c}]_{d}—,

—[OC(W)C(L)R⁷(CR⁸R⁹)ₑ]_{f}—

or

—{[NR¹⁰C(W)C(L)R¹¹(CR¹²R¹³)_{g}]ₕ[NR¹⁴C(W)(CR¹⁵R¹⁶)ᵢ]ⱼ}—,

wherein

a is an integer selected from 1 to 3;

b is an integer selected from 3 to 5;

c is an integer selected from 1 to 3;

d is an integer selected from 3 or 4;

e is an integer selected from 1 to 3;

f is an integer selected from 3 or 4;

g is an integer selected from 1 to 3;

h is an integer selected from 3 or 4;

i is an integer selected from 1 to 3;

j is an integer selected from 0 to 3;

k is an integer selected from 0 to 3;

m is an integer selected from 3 or 4;

L is a direct bond to [(CR¹⁷R¹⁸)ₖNH₂];

W is H₂ or O;

R¹, R², R³, R⁴, R⁵, R⁶, R⁷, R⁸, R⁹, R¹⁰, R¹¹, R¹², R¹³, R¹⁴, R¹⁵, and R¹⁶ are independently selected at each occurrence from H, C₁-C₆ alkyl, C₃-C₆ cycloalkyl, C₁-C₆ fluoroalkyl, C₁-C₆ alkenyl, C₃-C₆ cycloalkenyl, C₁-C₆ fluoroalkenyl, benzyl, fluorobenzyl, phenyl and fluorophenyl;

R¹⁷ and R¹⁸ are independently selected from H, C₁-C₁₀ alkyl substituted with 0-5 R²³, C₁-C₁₀ fluoroalkyl substituted with 0-5 R²³, C₂-C₁₀ alkenyl substituted with 0-5 R²³, C₂-C₁₀ fluoroalkenyl substituted with 0-5 R²³, aryl substituted with 0-5 R²³, C₇-C₁₆ alkaryl wherein the aryl is substituted with 0-5 R²³, and fluroaryl substituted with 0-5 R²³; or R¹⁷ and R¹⁸ may be taken together to form a C₃-C₁₀ cycloalkyl or C₃-C₁₀ cycloalkenyl optionally interrupted with C(O)NH, NH, NHC(O), NHC(O)NH, NHC(S)NH, O, S, S(O), S(O)₂, P(O)(OR²⁴), P(O)(OR²⁴)O or P(O)(NHR²⁴)O, or to form a ═CH—R^{22a} group, wherein R^{22a} is aryl substituted with 0-5 R²³, or heterocycle substituted by 0-5 R²³;

R²³ is selected from H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and

R²⁴ is selected from H, C₁-C₆ alkyl, C₃-C₆ cycloalkyl, C₁-C₆ fluoroalkyl, C₁-C₆ alkenyl, C₃-C₆ cycloalkenyl, C₁-C₆ fluoroalkenyl, benzyl, fluorobenzyl, phenyl, and fluorophenyl.

According to another embodiment of the present invention, pharmaceutical compositions are provided for treating pathological processes involving angiogenic neovasculature in a patient in need thereof containing the radiopharmaceutical compounds and conjugates of the present invention and a pharmaceutically acceptable carrier. In yet another embodiment of the present invention treatment methods are provided for pathological processes involving angiogenic neovasculature in a patient in need thereof, in which an effective amount of the aforesaid pharmaceutical composition is administered to the patient.

According to another embodiment of the present invention, radioactive imaging compositions are provided containing the radiopharmaceutical compounds and conjugates of the present invention and a pharmaceutically acceptable carrier. In yet another embodiment of the present invention, methods for radioactive imaging are provided in which an effective amount of the radioactive imaging compositions of the present invention are administered to a patient to be imaged sufficiently in advance thereto.

According to another embodiment of the present invention, magnetic resonance imaging compositions are provided containing the magnetic resonance imaging compounds and conjugates of the present invention and a pharmaceutically acceptable carrier. In yet another embodiment of the present invention, methods for magnetic resonance imaging are provided in which an effective amount of the magnetic resonance imaging compositions of the present invention are administered to a patient to be imaged sufficiently in advance thereto.

According to another embodiment of the present invention, X-ray and CT imaging compositions are provided containing the X-ray and CT imaging compounds and conjugates of the present invention and a pharmaceutically acceptable carrier. In yet another embodiment of the present invention, methods for X-ray and CT imaging are provided in which an effective amount of the X-ray or CT imaging compositions of the present invention are administered to a patient to be imaged sufficiently in advance thereto.

According to another embodiment of the present invention, compositions for treating heavy metal toxicity in a patient in need thereof are provided containing the polypodal chelant and conjugates thereof of the present invention and a pharmaceutically acceptable carrier. In yet another embodiment of the present invention, methods for treating heavy metal toxicity in a patient in need thereof are provided in which an effective amount of the aforesaid compositions of the present invention are administered to the patient.

Another embodiment of the present invention is diagnostic kits for the preparation of radiopharmaceuticals or radioactive, magnetic resonance, X-ray or CT imaging agents. Diagnostic kits of the present invention comprise one or more vials containing the sterile, non-pyrogenic, formulation comprised of a predetermined amount of a compound of the present invention, and optionally other components such as one or two ancillary ligands, reducing agents, transfer ligands, buffers, lyophilization aids, stabilization aids, solubilization aids and bacteriostats. The inclusion of one or more optional components in the formulation will frequently improve the ease of synthesis of the radiopharmaceutical by the practicing end user, the ease of manufacturing the kit, the shelf-life of the kit, or the stability and shelf-life of the radiopharmaceutical. The one or more vials that contain all or part of the formulation can independently be in the form of a sterile solution or a lyophilized solid.

### BRIEF DESCRIPTION OF THE DRAWINGS

FIG. 1 is a plot of the radiochemical purity of ¹¹¹In-DTPA-monoamide versus chelant/DTPA-monoamide ratio for a chelant of the present invention;

FIG. 2 is a plot of the radiochemical purity of ¹¹¹In-DTPA-bisamide versus chelant/DTPA-bisamide ratio for the same chelant;

FIG. 3 is a plot of the radiochemical purity of ¹⁵³Sm-DTPA-monoamide versus chelant/DTPA-monoamide ratio; and

FIG. 4 is a plot of the radiochemical purity of ¹⁵³Sm-DPTA-bisamide versus chelant/DTPA-bisamide ratio.

### DETAILED DESCRIPTION OF THE PREFERRED EMBODIMENTS

Polypodal chelants according to the present invention have the structure:

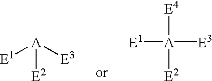

wherein A, E¹, E², E³ and E⁴ have the above-defined values. The spacer, A, is preferably R¹—C (in which R¹ is as defined above), N, P and P(O), or a macrocyclic group having the formula —[N(L)C(W)(CR⁵R⁶)_{c}]_{d}—, the variables for which also have the above-defined values. More preferably, A is N, P(O) or —[N(L)C(W)(CR⁵R⁶)_{c}]_{d}—. Even more preferably, A is N or P(O), and most preferably, A is N.

Each of R¹, and when A is —[N(L)C(W)(CR⁵R⁶)_{c}]_{d}, R⁵ and R⁶ is preferably independently selected from among H, C₁-C₆ alkyl, C₃-C₆ cycloalkyl, C₁-C₆ alkenyl, C₃-C₆ cycloalkyl, benzyl and phenyl. More preferably, R¹, R⁵ and R⁶ are selected from among H, C₁-C₆ alkyl, C₃-C₆ cycloalkyl, phenyl and benzyl. R⁵ and R⁶ are most preferably H, with c=2 and d=1 also being preferred.

E¹, E², E³ and E⁴ are preferably (CH₂)ₖ—NHCOCH₂NR²¹R²² or COCH₂NR²¹R²², wherein R²¹ and R²² are preferably independently selected at each occurrence from among H, C₁-C₁₀ alkyl substituted with 0-2 R²³, C₂-C₁₀ alkenyl substituted with 0-2 R²³, aryl substituted with 0-2 R²³ and C₇-C₁₆ alkaryl, wherein the aryl is substituted with 0-2 R²³, or R²¹ and R²² may be taken together to form a ═CH—R^{22a} group, wherein R^{22a} is aryl substituted with 0-5 R²³, or heterocycle substituted by 0-5 R²³, wherein each R²³ and R²⁴ has the above-defined value.

R²¹ and R²² are more preferably independently selected from among C₁-C₁₀ alkyl substituted with 0-2 R²³ and aryl substituted with 0-2 R²³, or R²¹ and R²² may be taken together to form a ═CH—R^{22a} group, wherein R^{22a} is aryl substituted with 0-5 R²³, or heterocycle substituted by 0-5 R²³. Most preferably, k is 2-3, R²¹ is independently selected from among CH₃, CH₂COOH and CH₂PO(OH)₂ and R²² is independently selected from among CH₂COOH and CH₂PO(OH)².

Each R²³ is preferably independently selected from among H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴. More preferably, each R²³ is independently selected from among OH, C₁-C₃ hydroxyalkyl, COOH, PO(OH)₂ and S(O)₂OH. Each R²⁴ is preferably independently selected from among H, C₁-C₆ alkyl, C₃-C₆ cycloalkyl, C₁-C₆ fluoroalkyl, C₁-C₆ alkenyl, C₃-C₆ cycloalkyl, benzyl and phenyl.

When R²¹ and R²² are taken together to form a ═CH—R^{22a} group, R^{22a} is preferably 2-hydroxyphenyl or 4-(2-methyl-3-hydroxy-5-hydroxymethyl)pyridyl.

Synthesis of Polypodal Chelants

The present invention provides polypodal chelants that can rapidly form highly stable metal chelates useful as diagnostic or therapeutic metalloradiopharmaceuticals, or magnetic resonance imaging contrast agents, or X-ray or CT contrast agents. In general, the polypodal chelants are divided into two classes: tripodal with three chelating arms and tetrapodal containing four chelating arms. The spacer A provides a 3-D chelant framework, and is selected from: R¹—C, R¹—Si, R¹—Ge, N, P, P(═O) or a macrocyclic group. The amide-nitrogen atoms in the chelant framework are not part of metal chelation. They serve as hydrogen-bonding units to keep polypodal strands in the right conformation for metal chelation. Likewise, the nitrogen heteroatoms incorporated into a preorganized macrocyclic backbone are used only as bridging atoms to connect the chelating arms with the cyclic chelant framework. The chelating groups are not limited to carboxylates and may contain groups such as phosphonate, phosphinate, hydroxamate, hydroxylethyl and hydroxyaryl.

The most critical step for the synthesis of these polypodal chelants involves preparation of intermediates (polyamines) with the 3-D chelant framework. Scheme I shows a general synthetic scheme for the preparation of protected polyamines. A[(CR¹⁷R¹⁸)ₖNH₂]ₘ, reacts with Lg—X—(CR¹⁹R²⁰)_{L}NH₂-Pg (Lg is a leaving group preferably selected from: Cl, Br, OC(═O)OR or NHS; X is selected from C(═O), S(═O)₂— or P(═O)(OR¹); and Pg is a protecting group preferably selected from Boc or CBZ). The deprotection of amino groups can be easily achieved by acid-catalyzed hydrolysis (Pg=Boc, for example) or metal-catalyzed hydrogenation (Pg=CBZ, for example) to give the corresponding free amines for further derivatization. Scheme I. General Synthetic Scheme for Protected Polyamines

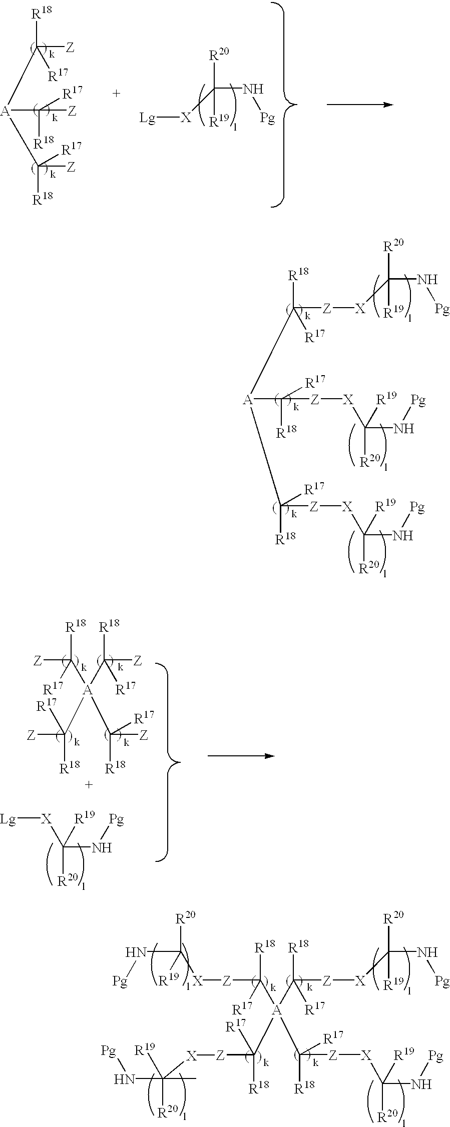

A[(CR¹⁷R¹⁸)ₖNH₂]ₘ is an important intermediate compound. Most of these tripodal or tetrapodal amines are either commercially available or can be readily prepared according to the literature methods. For example, tris(2-aminoethyl) amine and tris(3-aminopropyl)amine are available from Aldrich. 1,1,1-Tris(aminomethyl)ethane (TAME) and 1,2,3-triaminopropane can be prepared according to the procedure by Liu, et al (Inorg. Chem. 1993, 32, 4268-4276 and Inorg. Chem. 1993, 32, 1756-1783); 1,3,5-triaminocyclohexane by Bowen, T. et al (Bioorg. & Med. Chem. Lett. 1996, 6, 807-810); tris-endo-tricyclo-[5.2.1.0^{4,10}]decane-2,5,8-triamine by Aguilera, A. et al (Synthetic Commun. 1991, 21, 1643-1648); 2,2-bis(aminomethyl)-1,3-propanediamine by McAuley, A. et al (Can. J. Chem. 1989, 67, 1650-1656); 1,3,5-triamino-1,3,5-trideoxy-cis-inositol by Ghislett, M. et al (Helv. Chim. Acta 1992, 75, 2233-2251); germanium tetrahydrazide by Singh, P. R. et al (Nucl. Med. Biol. 1994, 21, 1115-1118); and trihydrazidophosphine oxide by Corlij, M. et al (J. Nucl. Biol. Med. 1992, 36, 296-300).

Examples of A[(CR¹⁷R¹⁸)ₖNH₂]ₘ include the following compounds:

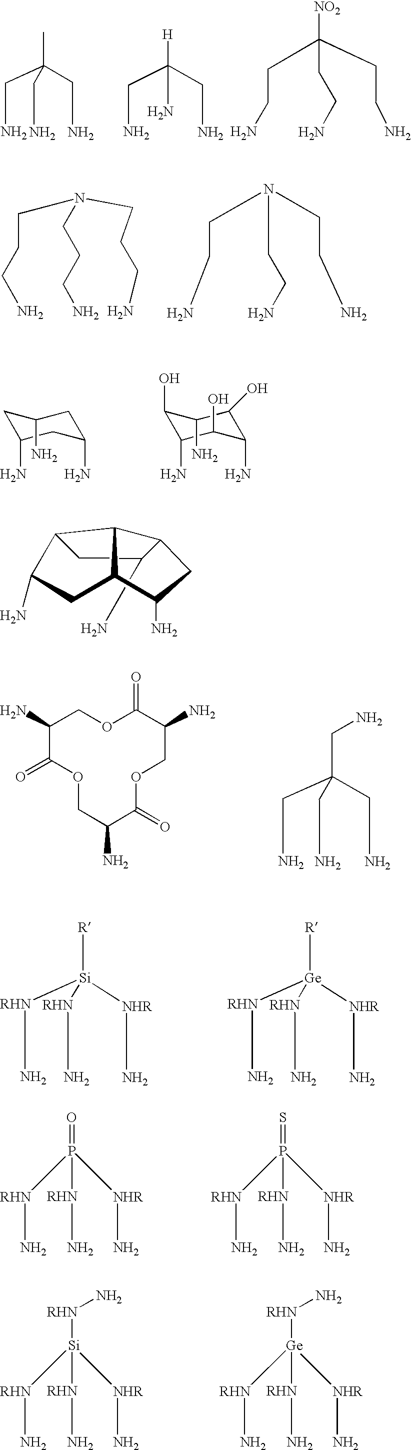

Lg is a leaving group and the choice of Lg is largely dependent on the group X (X═CO—, SO₂—, or PO₂R¹). If X is a carbonyl group, Lg—X will be selected from alkylcarbonyl chloride or bromide, N-hydroxy-succinamide ester, or an asymmetric anhydride. Scheme II shows the asymmetric anhydride approach for the synthesis of polypodal chelants.

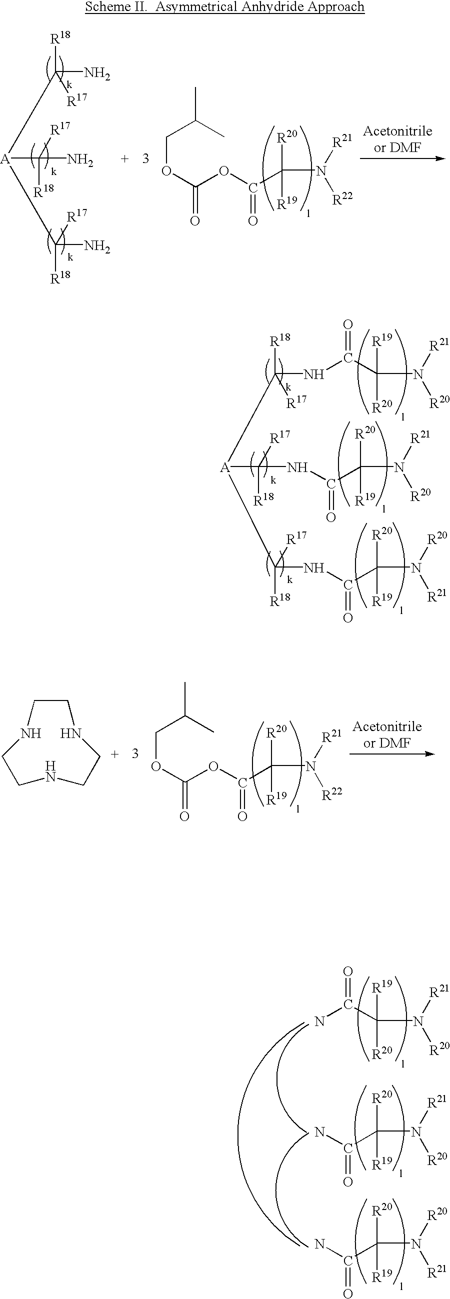

The alternative approach (Scheme IIb) uses N-hydroxy-succinimide ester (NHS-ester), which is reactive only towards primary amines. The reaction is usually carried out in acetonitrile or N,N-dimethylformamide (DMF). The protecting group (Pg) can be easily cleaved by acid hydrolysis (Pg=Boc, for example) or catalyzed hydrogenation (Pg=CBZ, for example) to give the corresponding free amine. Alkylation of the amino groups gives the corresponding polypodal chelant with two identical substituent groups. Alternatively, the polyamine intermediate can also functionalized by reacting with an aldehyde to form the Schiff base adduct. Reduction of the Schiff-base C═N bond and N-alkylation of the secondary amine will afford the polypodal chelant with two different substituent groups.

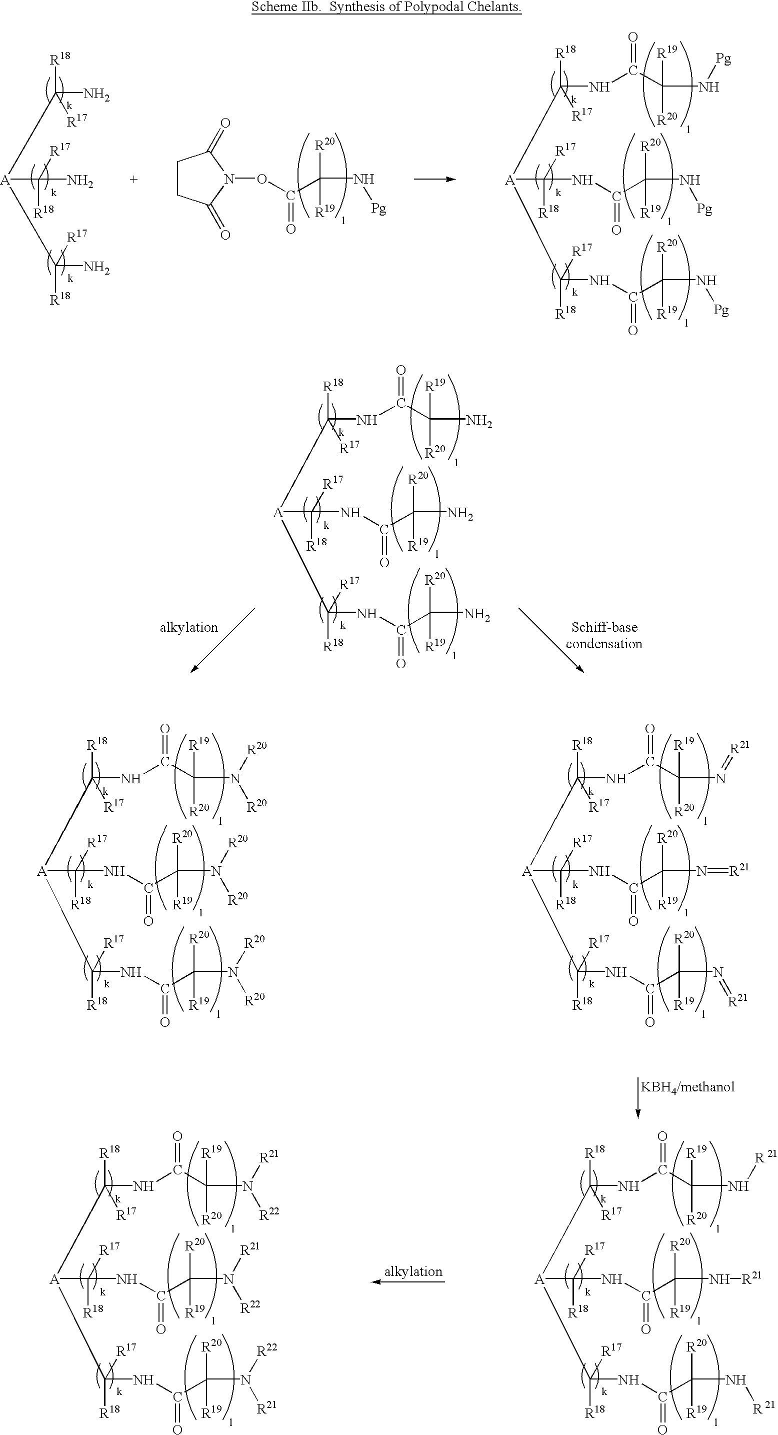

If X is a sulfonyl group, Lg—X is preferably sulfonyl chloride, which is reactive towards both primary and secondary amine groups (Scheme III). Reaction of chloride groups with a secondary amine gives the corresponding polypodal chelant with two identical or different substituent groups. This is particularly useful for polypodal chelants based on a macrocyclic chelant framework.

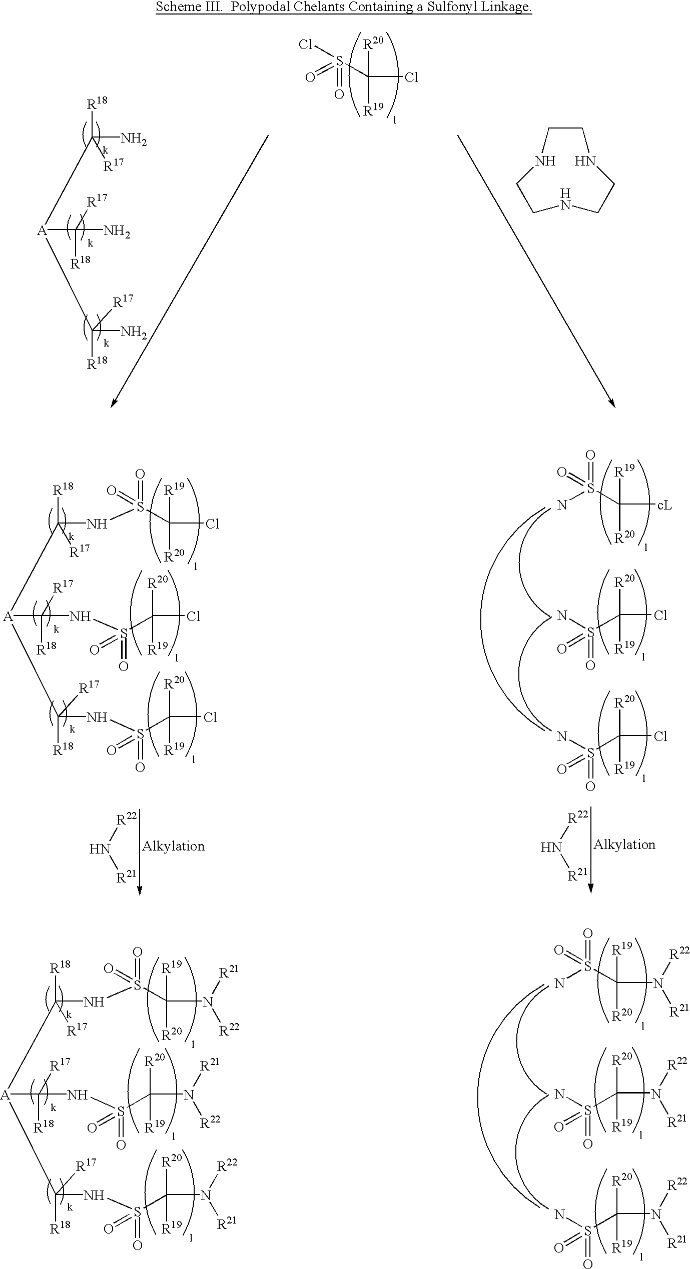

If X is P(═O)(OR¹), Lg—X is preferably a hydroxy-phosphoryl group, which can be readily activated by reacting with imidazole in the presence of a water-soluble catalyst such as EDC to give a phosphoryl-imidazole intermediate. The phosphoryl-imidazole intermediate reacts with a poly-podal amine to afford the protected polyamine (Scheme IV). The protecting group can be easily removed by acid hydro-lysis (Pg=Boc, for example) or hydrogenation (Pg=CBZ, for example) to give the free amine. Alkylation of the amino groups gives the corresponding polypodal chelant with two identical substituent groups. The polyamine can also react with an aldehyde to form the Schiff base. Reduction of the Schiff-base, followed by N-alkylation of the secondary amine produces the polypodal chelant with two different substituent groups.

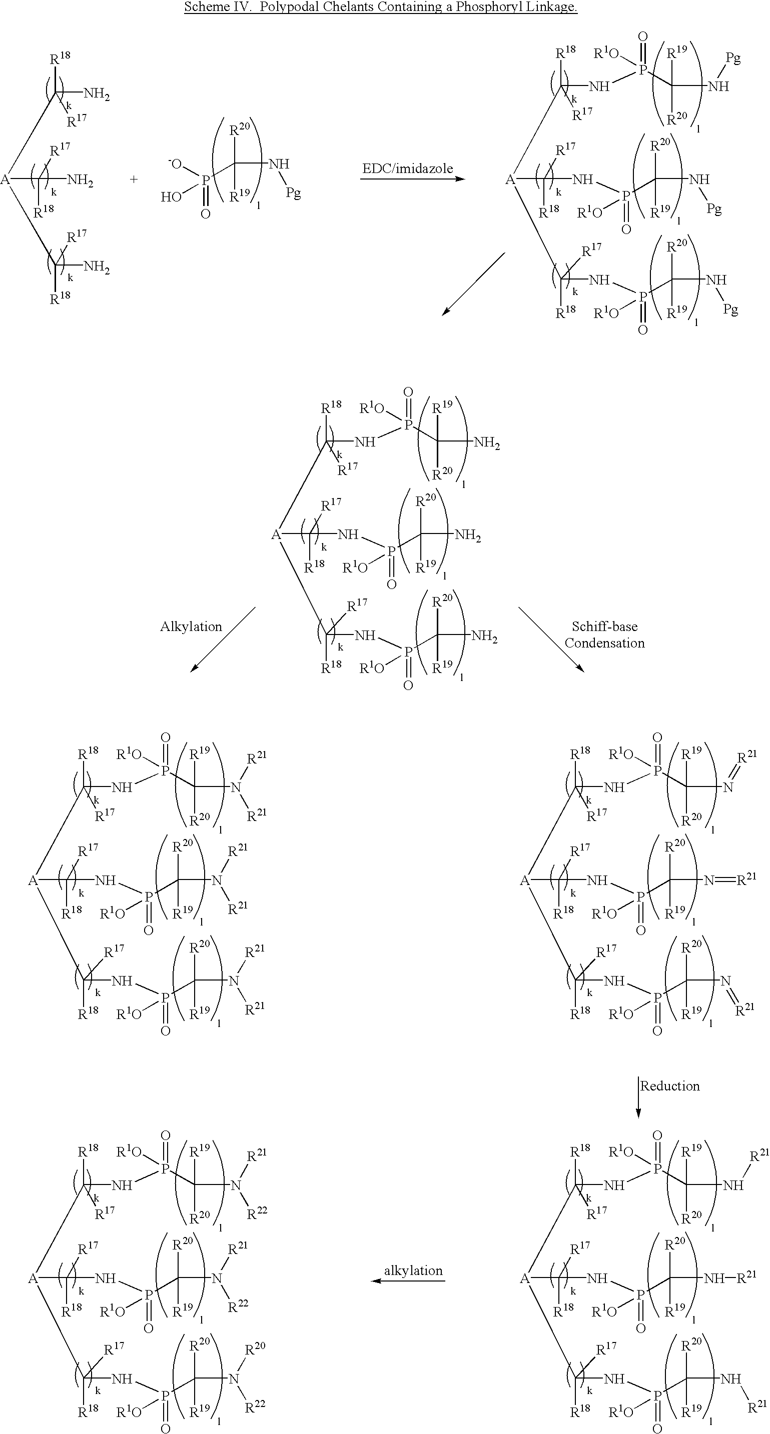

Definitions

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), for example, then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R⁹) occurs more than one time in any constituent or formula for a compound, its definition at each occurrence is independent of its definition at every other occurrence. Thus, for example, if a group is shown to be substituted with 0-2 R⁹, then said group may optionally be substituted with up to two R⁹ groups and R⁹ at each occurrence is selected independently from the definition of R⁹. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” is intended to include both branched and straight-chain saturated aliphatic hydrocarbon groups having the specified number of carbon atoms. Examples of alkyl include, but are not limited to, methyl, ethyl, n-propyl, i-propyl, n-butyl, s-butyl, t-butyl, n-pentyl, and s-pentyl. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl. “Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. “Cycloalkyl” is intended to include saturated ring groups, such as cyclopropyl, cyclobutyl, or cyclopentyl. Alkenyl” is intended to include hydrocarbon chains of either a straight or branched configuration and one or more unsaturated carbon-carbon bonds which may occur in any stable point along the chain, such as ethenyl and propenyl. “Alkynyl” is intended to include hydrocarbon chains of either a straight or branched configuration and one or more triple carbon-carbon bonds which may occur in any stable point along the chain, such as ethynyl and propynyl.

“Halo” or “halogen” as used herein refers to fluoro, chloro, bromo, and iodo; and “counterion” is used to represent a small, negatively charged species such as chloride, bromide, hydroxide, acetate, and sulfate.

As used herein, “carbocycle” or “carbocyclic residue” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

As used herein, the term “heterocycle” or “heterocyclic system” is intended to mean a stable 5- to 7-membered monocyclic or bicyclic or 7- to 10-membered bicyclic heterocyclic ring which is saturated partially unsaturated or unsaturated (aromatic), and which consists of carbon atoms and from 1 to 4 heteroatoms independently selected from the group consisting of N, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” or “heteroaryl” is intended to mean a stable 5- to 7-membered monocyclic or bicyclic or 7- to 10-membered bicyclic heterocyclic aromatic ring which consists of carbon atoms and from 1 to 4 heteroatoms independently selected from the group consisting of N, O and S. It is preferred that the total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridoxal, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Preferred heterocycles include, but are not limited to, pyridinyl, furanyl, thienyl, pyrrolyl, pyrazolyl, pyrrolidinyl, imidazolyl, indolyl, benzimidazolyl, 1H-indazolyl, oxazolidinyl, benzotriazolyl, benzisoxazolyl, oxindolyl, benzoxazolinyl, and isatinoyl. Also included are fused ring and spiro compounds containing, for example, the above heterocycles.

The term “amino acid” as used herein means an organic compound containing both a basic amino group and an acidic carboxyl group. Included within this term are α-amino acids, including natural amino acids (e.g., L-amino acids), modified and unusual amino acids (e.g., D-amino acids), as well as amino acids which are known to occur biologically in free or combined form but usually do not occur in proteins. Included within this term are modified and unusual amino acids,such as those disclosed in, for example, Roberts and Vellaccio (1983) The Peptides, 5: 342-429, the teaching of which is hereby incorporated by reference. Natural protein occurring amino acids include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-Cbz-protected amino acid, 2,4-diaminobutyric acid, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

The term “peptide” as used herein means a linear compound that consists of two or more amino acids (as defined herein) that are linked by means of a peptide bond. A “peptide” as used in the presently claimed invention is intended to refer to a moiety with a molecular weight of less than 10,000 Daltons, preferable less than 5,000 Daltons, and more preferably less than 2,500 Daltons. The term “peptide” also includes compounds containing both peptide and non-peptide components, such as pseudopeptide or peptidomimetic residues or other non-amino acid components. Such a compound containing both peptide and non-peptide components may also be referred to as a “peptide analog”.

A “pseudopeptide” or “peptidomimetic” is a compound which mimics the structure of an amino acid residue or a peptide, for example, by using linking groups other than amide linkages between the peptide mimetic and an amino acid residue (pseudopeptide bonds) and/or by using non-amino acid substituents and/or a modified amino acid residue. A “pseudopeptide residue” means that portion of an pseudopeptide or peptidomimetic that is present in a peptide.

The term “peptide bond” means a covalent amide linkage formed by loss of a molecule of water between the carboxyl group of one amino acid and the amino group of a second amino acid.

The term “pseudopeptide bonds” includes peptide bond isosteres which may be used in place of or as substitutes for the normal amide linkage. These substitute or amide “equivalent” linkages are formed from combinations of atoms not normally found in peptides or proteins which mimic the spatial requirements of the amide bond and which should stabilize the molecule to enzymatic degradation.

The term “non-peptide” refers to a compound in comprised of preferably less than three amide bonds in the backbone core compound or preferably less than three amino acids or amino acid mimetics.

A “diagnostic kit” or “kit” comprises a collection of components, termed the formulation, in one or more vials which are used by the practicing end user in a clinical or pharmacy setting to synthesize diagnostic radiopharmaceuticals. The kit provides all the requisite components to synthesize and use the diagnostic radiopharmaceutical except those commonly available to the practicing end user, such as water or saline for injection, a solution of the radio-nuclide, equipment for heating the kit during the synthesis of the radiopharmaceutical, if required, equipment necessary for administering the radiopharmaceutical to the patient such as syringes and shielding, and imaging equipment.

Buffers useful in the preparation of metallopharmaceuticals and in diagnostic kits for the preparation of said radiopharmaceuticals include but are not limited to phosphate, citrate, sulfosalicylate and acetate. A more complete list can be found in the United States Pharmacopeia.

A “transfer ligand” is a ligand that forms an intermediate complex with a metal ion that is stable enough to prevent unwanted side-reactions but labile enough to be converted to a metallopharmaceutical. The formation of the intermediate complex is kinetically favored while the formation of the metallopharmaceutical is thermodynamically favored. Transfer ligands useful in the preparation of metallopharmaceuticals and in diagnostic kits useful for the preparation of diagnostic radiopharmaceuticals include but are not limited to gluconate, glucoheptonate, mannitol, glucarate, N,N,N′,N′-ethylenediaminetetraacetic acid, pyrophosphate and methylenediphosphonate. In general, transfer ligands are comprised of oxygen or nitrogen donor atoms.

A “reducing agent” is a compound that reacts with a radionuclide, which is typically obtained as a relatively unreactive, high oxidation state compound, to lower its oxidation state by transferring electron(s) to the radionuclide, thereby making it more reactive. Reducing agents useful in the preparation of radiopharmaceuticals and in diagnostic kits useful for the preparation of said radiopharmaceuticals include but are not limited to stannous chloride, stannous fluoride, formamidine sulfinic acid, ascorbic acid, cysteine, phosphines, and cuprous or ferrous salts. Other reducing agents are described in PCT publication No. Wo 9422496 A1, which is incorporated herein by reference.

The phrase “pharmaceutically acceptable” is employed herein to refer to those compounds, materials, compositions, or dosage forms which are, within the scope of sound medical judgment, suitable for use in contact with the tissues of human beings and animals without excessive toxicity, irrita-tion, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers which release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent.

The coordination sphere of the radionuclide includes all the ligands or groups bound to the radionuclide. For a transition metal radionuclide, Mₜ, to be stable it typically has a coordination number (number of donor atoms) comprised of an integer greater than or equal to 4 and less than or equal to 9; that is there are 4 to 9 atoms bound to the metal and it is said to have a complete coordination sphere. The requisite coordination number for a stable radionuclide complex is determined by the identity of the radionuclide, its oxidation state, and the type of donor atoms.

Lyophilization aids useful in the preparation of diagnostic kits useful for the preparation of radiopharmaceuticals include but are not limited to mannitol, lactose, sorbitol, dextran, Ficoll, and polyvinylpyrrolidine (PVP).

Stabilization aids useful in the preparation of radiopharmaceuticals and in diagnostic kits useful for the preparation of said radiopharmaceuticals include but are not limited to ascorbic acid, cysteine, monothioglycerol, sodium bisulfite, sodium metabisulfite, gentisic acid, and inositol.

Solubilization aids useful in the preparation of radiopharmaceuticals and in diagnostic kits useful for the preparation of said radiopharmaceuticals include but are not limited to ethanol, glycerin, polyethylene glycol, propylene glycol, polyoxyethylene sorbitan monooleate, sorbitan monooleate, polysorbates, poly(oxyethylene)poly(oxypro-pylene)poly(oxyethylene) block copolymers (Pluronics) and lecithin. Preferred solubilizing aids are polyethylene glycol, and Pluronics.

Bacteriostats useful in the preparation of radiopharmaceuticals and in diagnostic kits useful for the preparation of said radiopharmaceuticals include but are not limited to benzyl alcohol, benzalkonium chloride, chlorbutanol, and methyl, propyl or butyl paraben.

The technetium and rhenium radiopharmaceuticals of the present invention can be easily prepared by admixing a salt of a radionuclide, a compound of the present invention, and a reducing agent, in an aqueous solution at temperatures from 0 to 100° C. The technetium and rhenium radionuclides are preferably in the chemical form of pertechnetate or perrhenate and a pharmaceutically acceptable cation. The pertechnetate salt form is preferably sodium pertechnetate such as obtained from commercial Tc-99m generators. The amount of pertechnetate used to prepare the radiopharmaceuticals of the present invention can range from 0.1 mCi to 1 Ci, or more preferably from 1 to 200 mCi.

The amount of the compounds of the present invention used to prepare the technetium and rhenium radiopharmaceuticals of the present invention can range from 0.01 μg to 10 mg, or more preferably from 0.5 μg to 200 μg. The amount used will be dictated by the amounts of the other reactants and the identity of the radiopharmaceuticals of the present invention to be prepared.

The metallopharmaceuticals of the present invention comprised of a metal of atomic number 21-31, 39-43, 44-50, 56-74, 76-80, 82-83, and 90 can be easily prepared by admixing a salt of a radionuclide and a reagent of the present invention, in an aqueous solution at temperatures from 0 to 100° C. These metals (radioisotopes, paramagnetic metals, and X-ray absorbing metals) are typically obtained as a dilute aqueous solution in a mineral acid, such as hydrochloric, nitric or sulfuric acid. The metals are combined with from one to about one thousand equivalents of the reagents of the present invention dissolved in aqueous solution. A buffer is typically used to maintain the pH of the reaction mixture between 3 and 10.

The total time of preparation will vary depending on the identity of the metal ion, the identities and amounts of the reactants and the procedure used for the preparation. The preparations may be complete, resulting in >80% yield of the metallopharmaceutical, in 1 minute or may require more time. If higher purity metallopharmaceuticals are needed or desired, the products can be purified by any of a number of techniques well known to those skilled in the art such as liquid chromatography, solid phase extraction, solvent extraction, dialysis or ultrafiltration.

The bio-targeted pharmaceuticals of the present invention have the formulae, (BM)_{d}-Lₙ—(Cₕ—X), and (BM)_{d}-Lₙ—(Cₕ—X¹)_{d′}, wherein BM represents a peptide, polypeptide, peptidomimetic, or non-peptide that binds to an object receptor or enzyme. For example, BM can be a peptide, polypeptide, peptidomimetic or non-peptide that binds to a receptor or enzyme expressed or up-regulated in angiogenic tumor vasculature. The value for d is 1-10, Lₙ represents an optional linking group, Cₕ represents a novel metal chelator of the present invention, d′ is 1-100, X represents a radioisotope, and X¹ represents paramagnetic metal ion.

The pharmaceuticals of the present invention can be synthesized by several approaches. One approach involves the synthesis of the targeting peptide, polypeptide, peptidomimetic or non-peptide moiety, BM, and direct attachment of one or more moieties, BM, to one or more metal chelators, Cₕ. Another approach involves the attachment of one or more moieties, BM, to the linking group, Lₙ, which is then attached to one or more metal chelators, Cₕ. Another approach, useful in the synthesis of pharmaceuticals wherein d is 1, involves the synthesis of the moiety, BM—Lₙ, together, by incorporating group bearing Lₙ into the synthesis of the peptide, polypeptide, peptidomimetic, or non-peptide. The resulting moiety, BM-Lₙ, is then attached to one or more metal chelators, Cₕ. Another approach involves the synthesis of a peptide, polypeptide, peptidomimetic, or non-peptide, BM, bearing a fragment of the linking group, Lₙ, one or more of which are then attached to the remainder of the linking group and then to one or more metal chelators, Cₕ.

The peptides, polypeptides, peptidomimetics and non-peptides, BM, optionally bearing a linking group, Lₙ, or a fragment of the linking group, can be synthesized using standard synthetic methods known to those skilled in the art. Preferred methods include but are not limited to those methods described below.

Generally, peptides, polypeptides, and peptidomimetics are elongated by deprotecting the alpha-amine of the C-terminal residue and coupling the next suitably protected amino acid through a peptide linkage using the methods described. This deprotection and coupling procedure is repeated until the desired sequence is obtained. This coupling can be performed with the constituent amino acids in a stepwise fashion, or condensation of fragments (two to several amino acids), or combination of both processes, or by solid phase peptide synthesis according to the method originally described by Merrifield (J. Am. Chem. Soc. 1963 85, 2149-2154), the disclosure of which is hereby incorporated by reference.

The peptides, polypeptides and peptidomimetics may also be synthesized using automated synthesizing equipment. In addition to the foregoing, procedures for peptide, polypeptide and peptidomimetic synthesis are described in Stewart and Young, “Solid Phase Peptide Synthesis”, 2nd ed, Pierce Chemical Co., Rockford, Ill. (1984); Gross, Meienhofer, Udenfriend, Eds., “The Peptides: Analysis, Synthesis, Biology, Vol. 1, 2, 3, 5, and 9, Academic Press, New York, (1980-1987); Bodanszky, “Peptide Chemistry: A Practical Textbook”, Springer-Verlag, New York (1988); and Bodanszky et al. “The Practice of Peptide Synthesis” Springer-Verlag, New York (1984), the disclosures of which are hereby incorporated by reference.

The coupling between two amino acid derivatives, an amino acid and a peptide, polypeptide or peptidomimetic, two peptide, polypeptide or peptidomimetic fragments, or the cyclization of a peptide, polypeptide or peptidomimetic can be carried out using standard coupling procedures such as the azide method, mixed carbonic acid anhydride (isobutyl chloroformate) method, carbodiimide (dicyclohexylcarbodiimide, diisopropylcarbodiimide, or water-soluble carbodiimides) method, active ester (p-nitrophenyl ester, N-hydroxysuccinic imido ester) method, Woodward reagent K method, carbonyldiimidazole method, phosphorus reagents such as BOP-C1, or oxidation-reduction method. Some of these methods (especially the carbodiimide) can be enhanced by the addition of 1-hydroxybenzotriazole. These coupling reactions may be performed in either solution (liquid phase) or solid phase.

The functional groups of the constituent amino acids or amino acid mimetics must be protected during the coupling reactions to avoid undesired bonds being formed. The protecting groups that can be used are listed in Greene, “Protective Groups in Organic Synthesis” John Wiley & Sons, New York (1981) and “The Peptides: Analysis, Synthesis, Biology, Vol. 3, Academic Press, New York (1981), the disclosure of which is hereby incorporated by reference.

The alpha-carboxyl group of the C-terminal residue is usually protected by an ester that can be cleaved to give the carboxylic acid. These protecting groups include: 1) alkyl esters such as methyl and t-butyl, 2) aryl esters such as benzyl and substituted benzyl, or 3) esters which can be cleaved by mild base treatment or mild reductive means such as trichloroethyl and phenacyl esters. In the solid phase case, the C-terminal amino acid is attached to an insoluble carrier (usually polystyrene). These insoluble carriers contain a group which will react with the carboxyl group to form a bond which is stable to the elongation conditions but readily cleaved later. Examples of which are: oxime resin (DeGrado and Kaiser (1980) J. Org. Chem. 45, 1295-1300) chloro or bromomethyl resin, hydroxymethyl resin, and aminomethyl resin. Many of these resins are commercially available with the desired C-terminal amino acid already incorporated.

The alpha-amino group of each amino acid must be protected. Any protecting group known in the art can be used. Examples of these are: 1) acyl types such as formyl, trifluoroacetyl, phthalyl, and p-toluenesulfonyl; 2) aromatic carbamate types such as benzyloxycarbonyl (Cbz) and substituted benzyloxycarbonyls, 1-(p-biphenyl)-1-methylethoxycarbonyl, and 9-fluorenylmethyloxycarbonyl (Fmoc); 3) aliphatic carbamate types such as tert-butyloxycarbonyl (Boc), ethoxycarbonyl, diisopropylmethoxycarbonyl, and allyloxycarbonyl; 4) cyclic alkyl carbamate types such as cyclopentyloxycarbonyl and adamantyloxycarbonyl; 5) alkyl types such as triphenylmethyl and benzyl; 6) trialkylsilane such as trimethylsilane; and 7) thiol containing types such as phenylthiocarbonyl and dithiasuccinoyl. The preferred alpha-amino protecting group is either Boc or Fmoc. Many amino acid or amino acid mimetic derivatives suitably protected for peptide synthesis are commercially available.

The alpha-amino protecting group is cleaved prior to the coupling of the next amino acid. When the Boc group is used, the methods of choice are trifluoroacetic acid, neat or in dichloromethane, or HCl in dioxane. The resulting ammonium salt is then neutralized either prior to the coupling or in situ with basic solutions such as aqueous buffers, or tertiary amines in dichloromethane or dimethylformamide. When the Fmoc group is used, the reagents of choice are piperidine or substituted piperidines in dimethylformamide, but any secondary amine or aqueous basic solutions can be used. The deprotection is carried out at a temperature between 0° C. and room temperature.

Any of the amino acids or amino acid mimetics bearing side chain functionalities must be protected during the preparation of the peptide using any of the above-identified groups. Those skilled in the art will appreciate that the selection and use of appropriate protecting groups for these side chain functionalities will depend upon the amino acid or amino acid mimetic and presence of other protecting groups in the peptide, polypeptide or peptidomimetic. The selection of such a protecting group is important in that it must not be removed during the deprotection and coupling of the alpha-amino group.

For example, when Boc is chosen for the alpha-amine protection the following protecting groups are acceptable: p-toluenesulfonyl (tosyl) moieties and nitro for arginine; benzyloxycarbonyl, substituted benzyloxycarbonyls, tosyl or trifluoroacetyl for lysine; benzyl or alkyl esters such as cyclopentyl for glutamic and aspartic acids; benzyl ethers for serine and threonine; benzyl ethers, substituted benzyl ethers or 2-bromobenzyloxycarbonyl for tyrosine; p-methylbenzyl, p-methoxybenzyl, acetamidomethyl, benzyl, or t-butylsulfonyl for cysteine; and the indole of tryptophan can either be left unprotected or protected with a formyl group.

When Fmoc is chosen for the alpha-amine protection usually tert-butyl based protecting groups are acceptable. For instance, Boc can be used for lysine, tert-butyl ether for serine, threonine and tyrosine, and tert-butyl ester for glutamic and aspartic acids.

Once the elongation of the peptide, polypeptide or peptidomimetic, or the elongation and cyclization of a cyclic peptide or peptidomimetic is completed all of the protecting groups are removed. For the liquid phase synthesis the protecting groups are removed in whatever manner as dictated by the choice of protecting groups. These procedures are well known to those skilled in the art.

When a solid phase synthesis is used to synthesize a cyclic peptide or peptidomimetic, the peptide or peptidomimetic should be removed from the resin without simultaneously removing protecting groups from functional groups that might interfere with the cyclization process. Thus, if the peptide or peptidomimetic is to be cyclized in solution, the cleavage conditions need to be chosen such that a free α-carboxylate and a free α-amino group are generated without simultaneously removing other protecting groups. Alternatively, the peptide or peptidomimetic may be removed from the resin by hydrazinolysis, and then coupled by the azide method. Another very convenient method involves the synthesis of peptides or peptidomimetics on an oxime resin, followed by intramolecular nucleophilic displacement from the resin, which generates a cyclic peptide or peptidomimetic (Osapay, Profit, and Taylor (1990) Tetrahedron Letters 43, 6121-6124). When the oxime resin is employed, the Boc protection scheme is generally chosen. Then, the preferred method for removing side chain protecting groups generally involves treatment with anhydrous HF containing additives such as dimethyl sulfide, anisole, thioanisole, or p-cresol at 0° C. The cleavage of the peptide or peptidomimetic can also be accomplished by other acid reagents such as trifluoromethanesulfonic acid/trifluoroacetic acid mixtures.

Unusual amino acids used in this invention can be synthesized by standard methods familiar to those skilled in the art (“The Peptides: Analysis, Synthesis, Biology, Vol. 5, pp. 342-449, Academic Press, New York (1981)). N-Alkyl amino acids can be prepared using procedures described in previously (Cheung et al., (1977) Can. J. Chem. 55, 906; Freidinger et al., (1982) J. Org. Chem. 48, 77 (1982)), which are incorporated herein by reference.

Additional synthetic procedures that can be used by one of skill in the art to synthesize the peptides, polypeptides and peptidomimetics targeting moieties are described in co-pending applications: U.S. application Ser. No. 08/943,659, U.S. application Ser. No. 09/281,474, U.S. application Ser. No. 09/466,588, U.S. application Ser. No. 09/465,300, U.S. application Ser. No. 09/466,582, U.S. application Ser. No. 60/182,627, and U.S. application Ser. No. 60/182,712, the contents of which are herein incorporated by reference.

The attachment of linking groups, Lₙ, to the peptides, polypeptides, peptidomimetics and non-peptide, BM; chelators, Cₕ, to the peptides, polypeptides, peptidomimetics, and non-peptides, W, or to the linking groups, Lₙ; and peptides, polypeptides, peptidomimetics, and non-peptides bearing a fragment of the linking group to the remainder of the linking group, in combination forming the moiety, (BM)_{d}—Lₙ, and then to the moiety Cₕ; can all be performed by standard techniques. These include, but are not limited to, amidation, esterification, alkylation, and the formation of ureas or thioureas. Procedures for performing these attachments can be found in Brinkley, M., Bioconjugate Chemistry 1992, 3(1), which is incorporated herein by reference.

The linking group Lₙ can serve several roles. First it provides a spacing group between the metal chelator, and the one or more of the peptides, polypeptides, peptidomimetics, or non-peptides, BM, so as to minimize the possibility that the moieties Cₕ—X, Cₕ—X¹, will interfere with the interaction of the recognition sequences of BM with the target receptors. The necessity of incorporating a linking group in a reagent is dependent on the identity of BM, Cₕ—X, and Cₕ—X¹. If Cₕ—X, and Cₕ—X¹, cannot be attached to BM without substantially diminishing its affinity for the receptors, then a linking group is used. A linking group also provides a means of independently attaching multiple peptides, polypeptides, peptidomimetics, and non-peptides, BM, to one group that is attached to Cₕ—X, or Cₕ—X¹.

The linking group also provides a means of incorporating a pharmacokinetic modifier into the pharmaceuticals of the present invention. The pharmacokinetic modifier serves to direct the biodistibution of the injected pharmaceutical other than by the interaction of the targeting moieties, BM, with the target receptors. A wide variety of functional groups can serve as pharmacokinetic modifiers, including, but not limited to, carbohydrates, polyalkylene glycols, peptides or other polyamino acids, and cyclodextrins. The modifiers can be used to enhance or decrease hydrophilicity and to enhance or decrease the rate of blood clearance. The modifiers can also be used to direct the route of elimination of the pharmaceuticals. Preferred pharmacokinetic modifiers are those that result in moderate to fast blood clearance and enhanced renal excretion.

For the diagnosis of thromboembolic disorders or atherosclerosis, BM is selected from the group including the cyclic IIb/IIIa receptor antagonist compounds described in U.S. Pat. No. 5,879,657; and the RGD containing peptides described in U.S. Pat. Nos. 4,578,079 and 4,792,525; published PCT Application Nos. WO 8905150, WO 8910135, WO 9101331, and WO 9115515, and by Ojima et. al., 204th Meeting of the Amer. Chem. Soc., 1992, Abstract 44. BM peptides also include the fibrinogen receptor antagonists described in published European Pat. Publication Nos. EP 410537, EP 410539, EP 410541, EP 422937, EP 422938, and EP 425212; the specific binding peptides and polypeptides described as IIb/IIIa receptor ligands, ligands for the polymerization site of fibrin, laminin derivatives, ligands for fibrinogen, or thrombin ligands in WO 93/23085 (excluding the technetium binding groups); the oligopeptides that correspond to the IIIa protein described in PCT WO90/00178; the peptides that are hirudin-based peptides described in WO90/03391; the peptides that are IIb/IIIa receptor ligands described in WO90/15818; the peptides that are thrombus, platelet binding or atherosclerotic plaque binding peptides described in WO92/13572 (excluding the technetium binding group) or GB 9313965.7; the peptides that are fibrin binding peptides described in U.S. Pat. Nos. 4,427,646 and 5,270,030; the peptides that are hirudin-based peptides described in U.S. Pat. No. 5,279,812; the peptides that are fibrin binding proteins described in U.S. Pat. No. 5,217,705; the guanine derivatives that bind to the IIb/IIIa receptor described in U.S. Pat. No. 5,086,069; the tyrosine derivatives described in published European Patent Application No. 478,328, and by Hartman et. al., J. Med. Chem., 35, 4640 (1992); or an oxidized low density lipoprotein (LDL).

For the diagnosis of infection, inflammation or transplant rejection, BM is selected from the group including the leukocyte binding peptides described in WO93/17719 (excluding the technetium binding group), WO92/13572 (excluding the technetium binding group) or U.S. Pat. No. 5,792,444; the chemotactic peptides described in published Eur. Pat. No. EP 398143 or A. Fischman et. al., Semin. Nuc. Med., 24, 154 (1994); the leukostimulatory agents described in U.S. Pat. No. 5,277,892; or the LTB4 antagonists described in co-pending U.S. patent application Ser. No. 08/943,659.

For the diagnosis of cancer, BM is selected from the group of somatostatin analogs described in published UK Patent Application No. GB 2225579, PCT Application No. WO90006949 or WO 94/00489, the selectin binding peptides described in WO 94/05269, the biological-function domains described in WO 93/12819, Platelet Factor 4 or the growth factors (PDGF, VEGF, EGF, FGF, TNF MCSF or the interleukins Il1-8).

BM may also be a compound that binds a receptor that is expressed or upregulated in angiogenic tumor vasculature. For targeting the VEGF receptors, Flk-1/KDR, Flt-1, and neuropilin-1, the targeting moieties are comprised of peptides, polypeptides or peptidomimetics that bind with high affinity to the receptors. For example, peptides comprised of a 23 amino acid portion of the C-terminal domain of VEGF have been synthesized which competitively inhibit binding of VEGF to VEGFR (Soker, et. al., J. Biol. Chem., 272, 31582-8 (1997)). Linear peptides of 11 to 23 amino acid residues that bind to the basic FGF receptor (bFGFR) are described by Cosic et. al., Mol. and Cell. Biochem., 130, 1-9 (1994). A preferred linear peptide antagonist of the bFGFR is the 16 amino acid peptide, Met-Trp-Tyr-Arg-Pro-Asp-Leu-Asp-Glu-Arg-Lys-Gln-Gln-Lys-Arg-Glu. Gho et. al. (Cancer Research, 57, 3733-40 (1997)) describe the identification of small peptides that bind with high affinity to the angiogenin receptor on the surface of endothelial cells. A preferred peptide is Ala-Gln-Leu-Ala-Gly-Glu-Cys-Arg-Glu-Asn-Val-Cys-Met-Gly-Ile-Glu-Gly-Arg, in which the two Cys residues form an intramolecular disulfide bond. Yayon et. al. (Proc. Natl. Acad. Sci, USA, 90, 10643-7 (1993)) describe other linear peptide antagonists of FGFR, identified from a random phage-displayed peptide library. Two linear octapeptides, Ala-Pro-Ser-Gly-His-Tyr-Lys-Gly and Lys-Arg-Thr-Gly-Gln-Tyr-Lys-Leu are preferred for inhibiting binding of bFGF to it receptor.

Targeting moieties for integrins expressed in tumor vasculature include peptides, polypeptides and peptidomimetics that bind to avB3, avB5, a5B1, a4B1, a1B1, and a2B2. Pierschbacher and Rouslahti (J. Biol. Chem., 262, 17294-8 (1987)) describe peptides that bind selectively to a5B1 and avB3. U.S. Pat. No. 5,536,814 describes peptides that bind with high affinity to the integrin a5B1. Burgess and Lim (J. Med. Chem., 39, 4520-6 (1996)) disclose the synthesis three peptides that bind with high affinity to avB3: cyclo[Arg-Gly-Asp-Arg-Gly-Asp], cyclo[Arg-Gly-Asp-Arg-Gly-D-Asp] and the linear peptide Arg-Gly-Asp-Arg-Gly-Asp. U.S. Pat. Nos. 5,770,565 and 5,766,591 disclose peptides that bind with high affinity to avB3. U.S. Pat. Nos. 5,767,071 and 5,780,426, disclose cyclic peptides that have an exocyclic Arg amino acid that have high affinity for avB3. Srivatsa et. al., (Cardiovascular Res., 36, 408-28 (1997)) describe the cyclic peptide antagonist for avB3, cyclo[Ala-Arg-Gly-Asp-Mamb]. Tran et. al., (Bioorg. Med. Chem. Lett., 7, 997-1002 (1997)) disclose the cyclic peptide cyclo[Arg-Gly-Asp-Val-Gly-Ser-BTD-Ser-Gly-Val-Ala] that binds with high affinity to avB3. Arap et. al. (Science, 279, 377-80 (1998)) describe cyclic peptides that bind to avB3 and avB5, Cys-Asp-Cys-Arg-Gly-Asp-Cys-Phe-Cys, and cyclo[Cys-Asn-Gly-Asp-Cys]. Corbett et. al. (Biorg. Med. Chem. Lett., 7, 1371-6 (1997)) describe a series of avB3 selective peptidomimetics. And Haubner et. al., (Angew. Chem. Int. Ed. Engl., 36, 1374-89 (1997)) disclose peptides and peptidomimetic avB3 antagonists obtained from peptide libraries.

Alternative targeting moieties for tumor vasculature include compounds that interact with receptor tyrosine kinases. Receptor tyrosine kinases (TKs) are membrane proteins, which play a key role in the transduction of mitogenic signals across the cell to the nucleus (Rewcastle, G. W. et al J. Med. Chem., 38, 3482-3487 (1995); Thompson, A. M. et al J. Med. Chem. 40, 3915-3925 (1997)). Of the many TKs that have been identified and characterized, those of the epidermal growth factor receptor (EGFR) family are particularly important, and have been implicated in a variety of ectopic cell proliferative processes. The over-expression of human EGF receptor is greatly amplified in several human tumors (Fry, D. W. Exp. Opin. Invest. Drugs, 3, 577-595 (1994); Jardines, L. et al Pathobiology, 61, 268-282 (1993)), accompanied by an over-phosphorylation of their protein targets. This increased phosphorylation of substrate tyrosine residues by oncogenic TK proteins is an essential step in the neoplastic transformation. Consequently, there has been great interest in developing inhibitors of TKs (TKIS) as anticancer drugs (Burke, T. R. Jr. Drugs Future, 17, 119-131 (1992); Chang, C. J. and Geahlen, R. J. Nat. Prod., 55, 1529-1560 (1992)). The over-expression of EGF receptors in tumor cells also provides the foundation for the development of diagnostic and therapeutic radiopharmaceuticals by attaching a chelator and a radionuclide onto the TK receptor ligand (tyrosine kinase inhibitor).

BM may also represent proteins, antibodies, antibody fragments, peptides, polypeptides, or peptidomimetics that bind to receptors or binding sites on other tissues, organs, enzymes or fluids. Examples include the β-amyloid proteins that have been demonstrated to accumulate in patients with Alzheimer's disease, atrial naturetic factor derived peptides that bind to myocardial and renal receptors, antimyosin antibodies that bind to areas of infarcted tissues, or nitroimidazole derivatives that localize in hypoxic areas in vivo.

### EXAMPLES

Acetic acid, ammonium hydroxide, Boc-glycine N-hydroxysuccinamide ester, t-butyl bromoacetate, isobutylchloroformate (IBCF), lanthanum nitrate hexahydrate, methyliminodiacetic acid (MIDA), nitrilotriacetic acid, potassium borohydride, anhydrous potassium dicarbonarte, salicyladehyde, 1,4,7-triazacyclononane ([9]aneN3), tris (2-aminoethyl) amine (tren), tris (3-aminopropyl)amine (trap), triethylamine, and trifluoroacetic acid, were purchased from either Aldrich or Sigma Chemical Co. and were used as received.

Instruments. ¹H NMR spectra were recorded on a 270 MHz Bruker spectrometer. The ¹H NMR data were reported as δ (ppm) relative to TMS. Electrospray MS analyses were performed using a VG Quattro mass spectrometer. LC-MS spectra were collected using a HP1100 LC/MSD system with API-electrospray interface.

### Example 1

Synthesis of Tren(Gly-Sal)₃

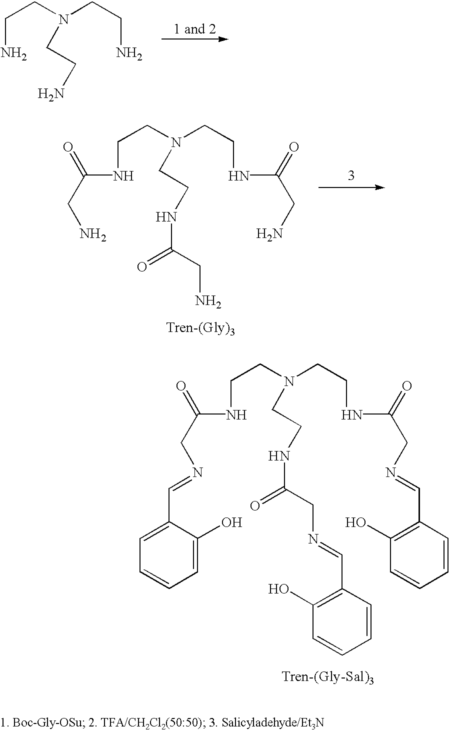

To a solution of Boc-Gly-OSu (8.16 g, 30 mmol) in acetonitrile (120 mL) was added tris(2-aminoethyl)amine (tren, 1.47 g, 10 mmol). The reaction mixture was stirred at room temperature for 5 h, and was then heated to reflux for 3 h. Solvent was removed on a rotary evaporator to give a gel-like thick liquid. The liquid was dissolved in dichloromethane (50 mL), followed by addition of trifluoroacetic acid (50 mL). The mixture was stirred at room temperature under nitrogen atmosphere for 2-3 h. Solvent and TFA were removed under reduced pressure to give a thick liquid, to which 30 mL of 4 N HCl was added. After stirring at room temperature for 30 min, volatiles were removed under educed pressure. The residue was dissolved in 30 mL of methanol. Upon addition of absolute ethanol (150 mL), a white precipitate was formed, and then became a gummy semi-solid. Solvents were decanted, and the residue was dried under vacuum to give a Tren-(Gly)₃ as its tetrahydrochloride salt. ¹H NMR (in D₂O: chemical shift δ in ppm relative to solvent signal): 3.95 (s, 6H, CH₂CONH); 3.77 (t, 8H, CH₂NH, J=6.2 Hz); 3.57 (t, 6H, NCH₂, J=6.4 Hz).

To a suspension of tren-(Gly)₃.4HCl (0.46 g, 1 mmol) in a mixture of methanol (5 mL) and ethanol (5 mL) was added salicylaldehyde (0.38 g, 3.3 mmol), and triethylamine (0.5 g, 5 mmol). The bright yellow solution was heated to reflux for 30 min, and was then cooled to room temperature. Slow addition of diethyl ether gave a bright yellow solid. The solid was collected by filtration, washed with diethyl ether, and dried under vacuum. The yield was 0.56 g (88%). ¹H NMR (in CDCl₃: chemical shift δ in ppm relative to solvent signal): 12.8 (bs, 3H, OH); 8.28 (s, 3H, CH═N); 6.80-7.10 (m, 12H, aromatic); 7.20 (t, 3H, NH); 4.26 (s, 6H, CH₂CONH); 3.42 (m, 6H, CONHCH₂); 2.53 (m, 6H, NCH₂). ESMS: m/z=630.3 (M+1, ES+; M=C₃₃H₃₉N₇O₆).

### Example 2

Synthesis of Tren(Gly-Pyridoxal)₃

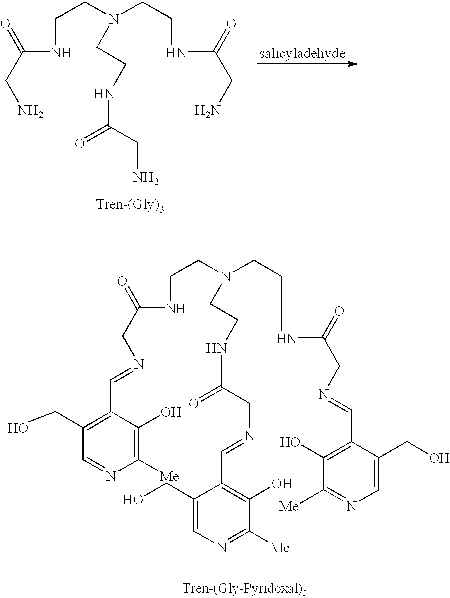

To a suspension of tren(Gly)₃.4HCl (0.46 g, 1 mmol) prepared according to example 1 in a mixture of methanol (10 mL) and was added pyridoxal hydrochloride (0.60 g, 3.0 mmol), and sodium acetate trihydrate (0.95 g, 7 mmol). The orange solution was heated to boil, and the solvent was evaporated slowly until ˜5 mL. The reaction mixture was cooled to room temperature. Slow addition of diethyl ether gave a yellow solid. The solid was separated, and was recrystallized from MeOH/Et₂O. The product was collected and dried under vacuum. The yield was 0.45 g (59%). ESMS: m/z=765.2 (M+1, ES+; M=C₃₆H₄₈N₁₀O₉)

### Example 3

Synthesis of Tren(Gly-HB)₃

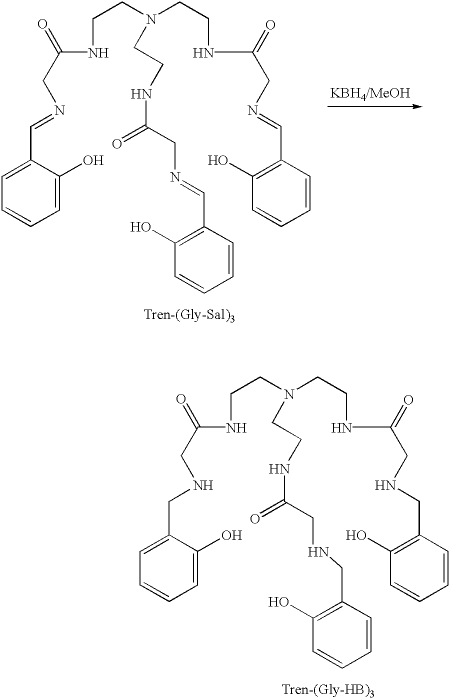

Tren(Gly-Sal)₃ (0.32, 0.5 mmole) prepared according to example 1 was dissolved in methanol (25 mL). Potassium borohydride was added in small portions until the bright yellow color disappeared. The solution (almost colorless) was stirred at room temperature for an additional 30 min. Volatiles were removed under reduce pressure. To the residue was added ammonium chloride (1.5 g) in water (10 mL), followed by extraction with dichloromethane (2×50 mL). Organic fractions were combined, and dried over anhydrous sodium sulfate. After filtration, the filtrate was evaporated to dryness to give a thick oil, which was dried under vacuum overnight to give a white powder. The yield was 0.18 g (56%). ¹H NMR (in CDCl₃: chemical shift δ in ppm relative to solvent signal): 6.80-7.15 (m, 12H, aromatic); 7.25 (t, 3H, NH); 4.50 (bs, 3H, OH); 3.81 (s, 6H, PhCH₂); 3.27 (m, 6H, CONHCH₂); 3.19 (s, 6H, CH₂CONH); 2.53 (m, 6H, NCH₂). ESMS: m/z=636.3 (M+1, ES+; M=C₃₃H₄₅N₇O₆).

### Example 4

Synthesis of Tren(NTA)₃.4HCl

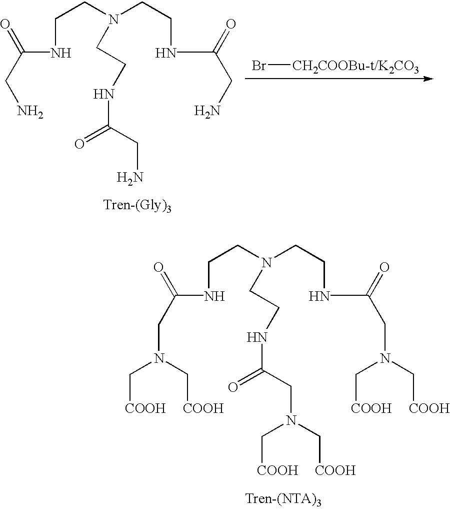

To a suspension of tren(Gly)₃.4HCl (0.95 g, 2.1 mmol) in DMF (100 mL) was added 5 g of potassium dicarbonate and t-butyl bromoacetate (7 g, 36 mmol). The mixture was stirred at room temperature for 24 hours. The solid was filtered out and discarded, and the filtrate was evaporated to dryness to give a brownish oil, which was re-dissolved in dichloromethane (50 mL). The solution was filtered. Solvent was removed under reduced pressure to give an oily residue. To the residue was added 10 mL of concentrated HCl and 20 mL of water. The mixture was stirred at room temperature for 30 min. Volatiles were removed to give a brownish solid, which was dried under vacuum to afford Tren(NTA)₃ at its tetrahydrochloride salt. ¹H NMR (in D₂O, chemical shift δ in ppm relative to solvent signal): 4.30 (s, 6H, NHCH₂CONH); 4.22 (s, 12H, CH₂COOH); 3.70 (t, 6H, CONHCH₂); 3.52 (t, 6H, NCH₂). ESMS: m/z=666.3 (M+1, ES+; M=C₂₄H₃₉N₇O₁₅).

The product is very hygroscopic, and can be easily converted to its sodium salt form by reacting with NaOH. It was found that the hexasodium salt of Tren(NTA)₃ is identical to that prepared from asymmetric NTA-IBF anhydride.

### Example 5

Synthesis of Hexasodium Salt of Tren(NTA)₃

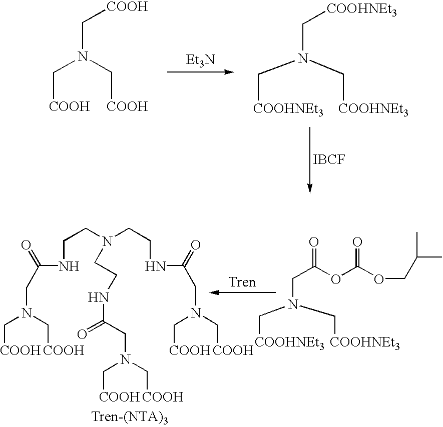

To suspension of nitrilotriacidic acid (1.15 g, 6 mmol) in dry acetonitrile (100 mL) was added triethylamine (1.9 g, 19 mmol). The resulting mixture was stirred under nitrogen atmosphere until a clear and homogenous solution was obtained. The solution was stirred at room temperature for 30 min, and was then cooled down to −35° C. Isobutylchloro-formate (0.82 g, 6 mmol) was added to give a white slurry. The slurry was stirred at −35° C for 20 min, warmed up to 0-5° C., and stirred at 0-5° C. for another 15 min. To the reaction mixture was added 2 mmol of tren (0.30 g, 2 mmol). The resulting mixture was stirred at 0-5° C. for 2 h, and then heated to reflux for another 2 hours. Solvent was removed under reduced pressure. To the residue was added 5 mL of 5 N NaOH solution, followed by addition of 50 mL of methanol and 50 mL of acetone to give a white precipitate. The precipitate was collected by filtration, washed with methanol, and dried under vacuum overnight. The yield was usually ≧80%. Recrystallization can be achieved by dissolving the sample in 5 mL of water. Slow addition of methanol (50 mL) gives a white solid. The solid was separated, washed with methanol, and dried under vacuum. The yield was 1.5 g. ¹H NMR (in D₂O, chemical shift δ in ppm relative to solvent signal): 3.40 (t, 6H, CONHCH₂); 3.38 (s, 6H, CH₂CONH); 3.30 (s, 12H, CH₂COONa); 2.53 (t, 6H, NCH₂). ESMS: m/z=664.2 (M−1, ES−; M=C₂₄H₃₉N₇O₁₅).

### Example 6

Synthesis of Trap(Gly-Sal)₃

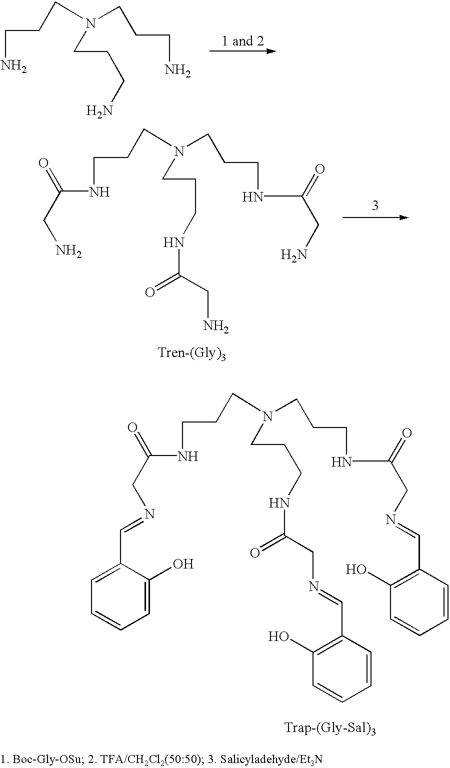

To a solution of Boc-Gly-OSu (16.3 g, 60 mmol) in acetonitrile (250 mL) was added tris(3-aminopropyl)amine (trap, 3.95 g, 20 mmol). The reaction mixture was stirred at room temperature for 2 h, and was then heated to reflux for 3 h. Solvent was removed on a rotary evaporator to give a thick liquid. The liquid residue was dissolved in dichloromethane (50 mL), followed by addition of trifluoroacetic acid (50 mL). The mixture was stirred at room temperature under nitrogen atmosphere for 2-3 h. The solvent and TFA were removed under reduced pressure to give a thick liquid, to which 40 mL of 4 N HCl was added. After stirring at room temperature for 30 min, volatiles were removed under reduced pressure. The residue was dissolved in 50 mL of methanol. Upon addition of acetone (100 mL) a white precipitate was formed, and then became a gummy semi-solid. Solvents were decanted, and the residue was dried under vacuum to give Trap(Gly)₃ as its tetrahydrochloride salt. ¹H NMR (in D₂O: chemical shift δ in ppm relative to solvent signal): 3.92 (s, 6H, CH₂CONH); 3.45 (m, 6H, CONHCH₂); 3.30 (m, 6H, NCH₂); 2.05 (m, 6H, CH₂).

To a suspension of trap-(Gly)₃.4HCl (0.50 g, 1 mmol) prepared according to example 5 in methanol (5 mL) and was added salicylaldehyde (0.38 g, 3.3 mmol), and sodium acetate trihydrate (0.55 g, 4 mmol). The bright yellow solution was heated to reflux for 30 min, and was then cooled to room temperature. Slow addition of diethyl ether gave a bright yellow solid. The solid was collected by filtration, washed with diethyl ether, and dried under vacuum. The yield was 0.38 g (57%). ESMS: m/z=672.3 (M+1, ES+; M=C₃₃H₃₉N₇O₆).

### Example 7

### Synthesis of Hexasodium Salt of Trap(NTA)₃

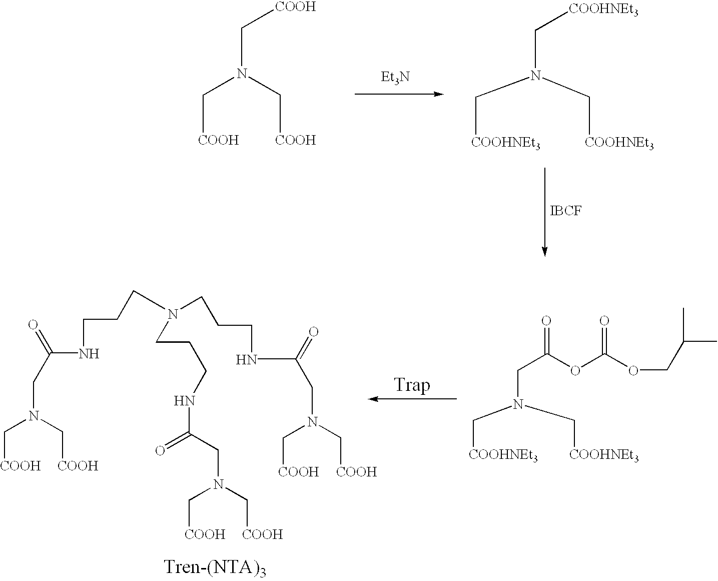

Trap(NTA)₃ was prepared in a similar fashion to that in example x using 2 mmol of tris(3-aminopropyl)amine (trap). Solvent was removed under reduced pressure. To the residue was added 5 mL of 5 N NaOH solution, followed by addition of 50 mL of methanol and 50 mL of acetone to give a white precipitate. The precipitate was separated by filtration and redissolved in minimum amount of methanol. Slow addition of acetone (50 mL) gives a white solid. The solid was separated, washed with acetone, and dried under vacuum. The yield was 1.25 g. ¹H NMR (in D₂O, chemical shift δ in ppm relative to solvent signal): 3.40 (d, 6H, CONHCH₂); 3.29 (s, 6H, CH₂CONH); 3.25 (s, 12H, CH₂COONa); 2.59 (m, 6H, NCH₂); 1.76 (m, 6H, CH₂). ESMS: m/z=708.3 (M+1, ES+; M=C₂₇H₄₅N₇O₁₅).

### Example 8

Synthesis of Hexasodium Salt of [9]aneN3 (NTA)₃

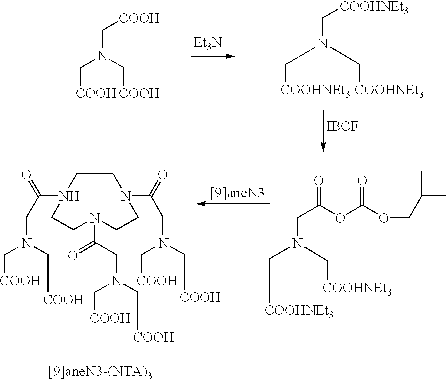

[9]aneN3 (NTA)₃ was prepared in a same way as that in Example 5 using 2 mmol of 1,4,7-trazacyclononane ([9]aneN3). Solvent was removed under reduced pressure. To the residue was added 5 mL of 5 N NaOH solution, followed by addition of 50 mL of methanol and 50 mL of acetone to give a white precipitate. The precipitate was separated by filtration, washed with acetone, and dried under vacuum. The yield was 0.78 g. ¹H NMR (in D₂O, chemical shift δ in ppm relative to solvent signal): 3.50-3.80 (m, 12H, NCH₂CH₂N) ; 3.40(s, 6H, CH₂CONH); 3.28 (m, 12H, CH₂COONa). ESMS: m/z=647.3 (M−1, ES−, M=C₂₄H₃₆N₆O₁₅)

### Example 9

Synthesis of Trisodium Salt of Tren(MIDA)₃

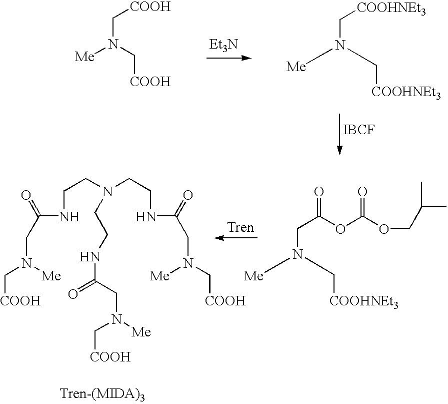

To suspension of methyliminodiacetic acid (1.76 g, 12 mmol) in dry acetonitrile (100 mL) was added triethylamine (2.5 g, 25 mmol). The resulting mixture was stirred under nitrogen atmosphere until a clear and homogenous solution was obtained. The solution was stirred at room temperature for 30 min, and was then cooled down to −35° C. Isobutyl-chloroformate (1.63 g, 12 mmol) was added to give a white slurry. The slurry was stirred at −35° C. for 20 min, warmed up to 0-5° C., and stirred at 0-5° C. for another 15 min. To the reaction mixture was added tren (0.58 g, 4 mmol). The resulting mixture was stirred at 0-5° C. for 2 h, and then heated to reflux for another 2 hours. Solvent was removed under reduced pressure to give a white solid residue, to which 5 mL of 5 N NaOH solution was added. Addition of 20 mL of methanol and 50 mL of acetone gave a sticky semisolid. Solvents were decanted and discarded. The residue was redissolved in water (5 mL), and to the resulting solution was added methanol (20 mL) and acetone (50 mL) to give a white solid. The solid was separated, and dried under vacuum. The yield was 2.6 g. ¹H NMR (in D₂O, chemical shift δ in ppm relative to solvent signal): 3.42 (t, 6 H, CONHCH₂); 3.27 (s, 6H, CH₂CONH); 3.16 (s, 6H, CH₂COONa); 2.82 (t, 6H, NCH₂); 2.34 (s, 9H, CH₃).

### Example 10

Synthesis of Trisodium Salt of Trap(MIDA)₃

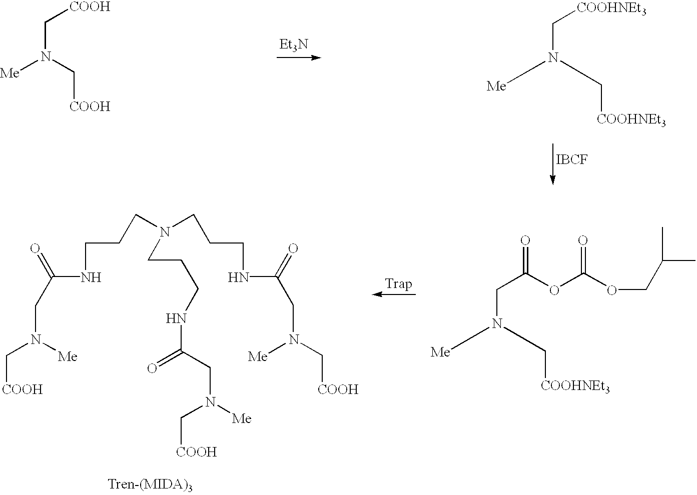

Trap(MIDA)₃ was prepared in a similar way to that for Tren-(MIDA)₃ using 4.0 mmole of trap. After solvent was removed under reduced pressure, 5 mL of 5 N NaOH solution was added to the residue, followed by addition of 20 mL of methanol and 50 mL of acetone to give a sticky semisolid. Solvents were decanted and discarded. To the residue was added 20 mL of methanol to give a cloudy solution. Addition of acetone (50 mL) gave a white solid. The solid was separated, and dried under vacuum. The yield was 1.5 g. ¹H NMR (in D₂O, chemical shift δ in ppm relative to solvent signal): 3.30 (t, 6 H, CONHCH₂); 3.25 (s, 6H, CH₂CONH); 3.16 (s, 6H, CH₂COONa); 2.57 (t, 6H, NCH₂); 2.34 (s, 9H, CH₃); 1.76 (m, 6H, CH₂).

### Example 11

Synthesis of Na₃[La-Tren(NTA)₃]

To a solution of La(NO₃)₃•6H₂O (220 mg, 0.5 mmol) in water (5 mL) was added Tren(NTA)₃ in its hexasodium form (400 mg, 0.5 mmol) in water (3 mL). The resulting mixture was heated to boil. The pH was adjusted to pH 5 using acetic acid and the white precipitate disappeared. The pH was then adjusted to about 10 using 1 N NaOH solution to give a clear solution. The reaction mixture was refluxed for 20 min, and was then concentrated to ˜3 mL. Addition of methanol (7 mL) gave a white precipitate, which was separated by filtration, washed with methanol, and dried under vacuum overnight. The yield was 250 mg. ¹H NMR (in D₂O, chemical shift δ in ppm relative to solvent signal): 3.60 (m, 6H, CONHCH₂); 3.50 (s, 6H, CH₂CONH); 3.40 (s, 12H, CH₂COONa); 2.76 (t, 6H, NCH₂). ESMS: m/z=867.9 (M−1, ES−, M=C₂₄H₃₉N₇O₁₅LaNa₃), 844.0 (M−Na), 822.3 (M−2Na+H), and 800.1 (M−3Na+2H).

### Example 12

Synthesis of ⁹⁰Y complex of Tren(NTA)₃

To a clean 5 mL vial containing 1.0 mL of Tren(NTA)₃ solution (4 mg/mL in 0.5 M NH₄OAc, pH=7.5) was added 2.5 μL of ⁹⁰YCl₃ solution (˜3 mCi) in 0.05 N HCl. The reaction mixture was kept at room temperature 20-30 min, and was then analyzed by an ITLC method, which uses Gelman Sciences silica gel paper strips and mixture of saline and acetone (50:50=v:v). The radiolabeling yield was 78.0%.

### Example 13

Synthesis of ⁹⁰Y complex of Trap(NTA)₃

To a clean 5 mL vial containing 1.0 mL of Trap(NTA)₃ solution (4 mg/mL in 0.5 M NH₄OAc, pH=7.5) was added 2.5 μL of ⁹⁰YCl₃ solution (˜3 mCi) in 0.05 N HCl. The reaction mixture was kept at room temperature 20-30 min, and was then analyzed by an ITLC method, which uses Gelman Sciences silica gel paper strips and mixture of saline and acetone (50:50=v:v). The radiolabeling yield was 72.0%.

### Example 14

Synthesis of ⁹⁰Y complex of [9]aneN₃(NTA)₃

To a 5 mL vial containing 1.0 mL of [9]aneN₃(NTA)₃ solution (4 mg/mL in 0.5 M NH₄OAc, pH=7.5) was added 2.5 μL of ⁹⁰YCl₃ solution (˜3 mCi) in 0.05 N HCl. The reaction mixture was kept at room temperature 20-30 min, and was then analyzed by an ITLC method, which uses Gelman Sciences silica gel paper strips and mixture of saline and acetone (50:50=v:v). The radiolabeling yield was 87%.

Chelant Challenging Experiment

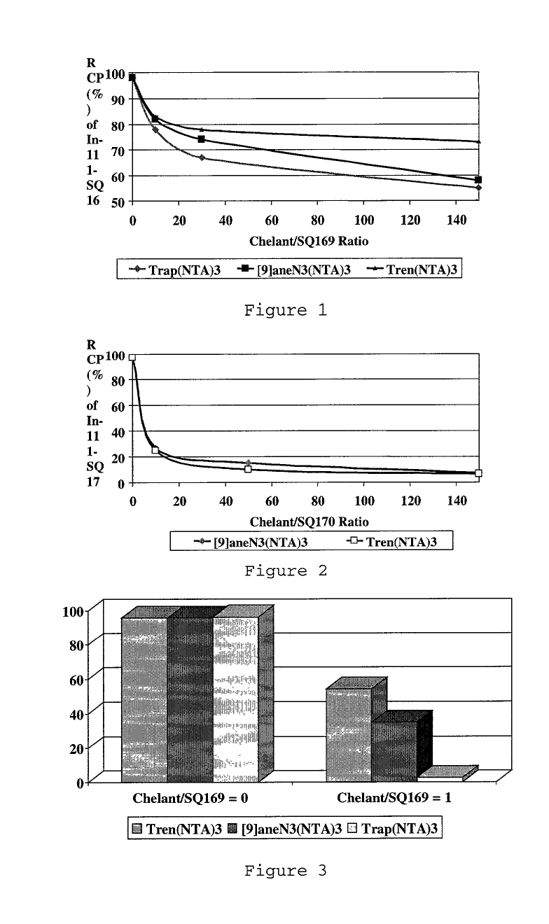

To determine the relative stability of the ¹¹¹In complexes of new chelants, a chelant challenging experiment was performed, in which the complexes ¹¹¹In-SQ169 (DTPA-monoamide) and ¹¹¹In-SQ170 (DTPA-bisamide) were first prepared and then challenged by adding a chelant at various concentrations. The reaction of ¹¹¹In complexes with a chelant was performed by heating the reaction mixture at 80° C. for 30 min in order to complete the ligand exchange. FIGS. 1 and 2 show plots of radiochemical purity (RCP) of ¹¹¹In complex versus the chelant/SQ169 or chelant/SQ170 ratio. This experiment shows that new chelants described are able to form ¹¹¹In complexes; but the stability of ¹¹¹In complexes of Tren(NTA)₃, Trap(NTA)₃, and [9]aneN₃(NTA)₃ is lower than that of both ¹¹¹In-DTPA-monoamide and ¹¹¹In-DTPA-bisamide complexes.

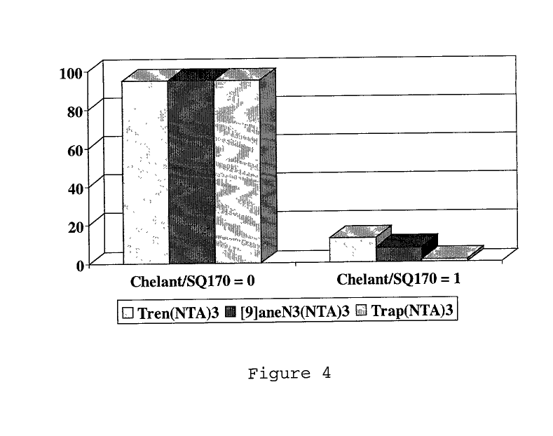

Due to the size difference, In complexes may have different structures from those of their lanthanide counterparts. In order to predict the stability of lanthanide complexes, we used ¹⁵³Sm as an example for lanthanide radionuclide. In this experiment, an 1:1 chelant/SQ169 or chelant/SQ170 ratio was used in order to determine the relative stability of ¹⁵³Sm complexes of Tren(NTA)₃, Trap(NTA)₃, and [9]aneN₃(NTA)₃. Complexes ¹⁵³Sm-SQ169 and ¹⁵³Sm-SQ170 were first prepared. The resulting solution was divided into three separate reaction vials, to which equal molar concentration of a chelant was added. These three vials were heated at 80° C. for 30 min and the resulting reaction mixtures were characterized by HPLC. FIGS. 3 and 4 show histograms of RCP for ¹⁵³Sm-SQ169 (top) and ¹⁵³Sm-SQ170 (bottom) versus the chelant/SQ169 and chelant/SQ170 ratio, respectively. It is quite clear that the stability of ¹⁵³Sm complexes of Tren(NTA)₃, Trap(NTA)₃, and [9]aneN₃(NTA)₃ is better than that of both ¹⁵³Sm-DTPA-bisamide and ¹⁵³Sm-DTPA-monoamide. This experiment clearly demonstrated that the new chelants described in this invention is highly selective for lanthanide metal ions over smaller In³⁺ cation.

Utility

The diagnostic radiopharmaceuticals are administered by intravenous injection, usually in saline solution, at a dose of 1 to 100 mCi per 70 kg body weight, or preferably at a dose of 5 to 50 mCi. Imaging is performed using known procedures.

The therapeutic radiopharmaceuticals are administered by intravenous injection, usually in saline solution, at a dose of 0.1 to 100 mCi per 70 kg body weight, or preferably at a dose of 0.5 to 5 mCi per 70 kg body weight.

The magnetic resonance imaging contrast agents of the present invention may be used in a similar manner as other MRI agents as described in U.S. Pat. Nos. 5,155,215; 5,087,440; Margerstadt et al., Magn. Reson. Med., 1986, 3, 808; Runge et al., Radiology, 1988, 166, 835; and Bousquet et al., Radiology, 1988, 166, 693. Generally, sterile aqueous solutions of the contrast agents are administered to a patient intravenously in dosages ranging from 0.01 to 1.0 mmoles per kg body weight.

For use as X-ray contrast agents, the compositions of the present invention should generally have a heavy atom concentration of 1 mM to 5 M, preferably 0.1 M to 2 M. Dosages, administered by intravenous injection, will typically range from 0.5 mmol/kg to 1.5 mmol/kg, preferably 0.8 mmol/kg to 1.2 mmol/kg. Imaging is performed using known techniques, preferably X-ray computed tomography.

Other features of the invention will become apparent in the course of the following descriptions of exemplary embodiments which are given for illustration of the invention and are not intended to be limiting thereof.

The pharmaceuticals of the present invention are useful for imaging angiogenic tumor vasculature in a patient or for treating cancer in a patient. The radiopharmaceuticals of the present invention comprised of a gamma emitting isotope are useful for imaging of pathological processes involving angiogenic neovasculature, including cancer, diabetic retinopathy, macular degeneration, restenosis of blood vessels after angioplasty, and wound healing. The radiopharmaceuticals of the present invention comprised of a beta, alpha or Auger electron emitting isotope are useful for treatment of pathological processes involving angiogenic neovasculature, by delivering a cytotoxic dose of radiation to the locus of the angiogenic neovasculature. The treatment of cancer is affected by the systemic administration of the radiopharmaceuticals resulting in a cytotoxic radiation dose to tumors.

The compounds of the present invention comprised of one or more paramagnetic metal ions selected from gadolinium, dysprosium, iron, and manganese, are useful as contrast agents for magnetic resonance imaging (MRI) of pathological processes involving angiogenic neovasculature.

The compounds of the present invention comprised of one or more heavy atoms with atomic number of 20 or greater are useful as X-ray contrast agents for X-ray imaging of pathological processes involving angiogenic neovasculature.

Representative compounds of the present invention were tested in the following in vitro and in vivo assays and models and were found to be active.

Immobilized Human Placental aᵥb₃ Receptor Assay

The assay conditions were developed and validated using [I-125]vitronectin. Assay validation included Scatchard format analysis (n=3) where receptor number (Bmax) and Kd (affinity) were determined. Assay format is such that compounds are preliminarily screened at 10 and 100 nM final concentrations prior to IC50 determination. Three standards (vitronectin, anti-aᵥb₃ antibody, LM609, and anti-aᵥb₅, P1F6) and five reference peptides have been evaluated for IC50 determination. Briefly, the method involves immobilizing previously isolated receptors in 96 well plates and incubating overnight. The receptors were isolated from normal, fresh, non-infectious (HIV, hepatitis B and C, syphilis, and HTLV free) human placenta. The tissue was lysed and tissue debris removed via centrifugation. The lysate was filtered. The receptors were isolated by affinity chromatography using the immobilized aᵥb₃ antibody. The plates are then washed 3× with wash buffer. Blocking buffer is added and plates incubated for 120 minutes at room temperature. During this time, compounds to be tested and [I-125]vitronectin are premixed in a reservoir plate. Blocking buffer is removed and compound mixture pipetted. Competition is carried out for 60 minutes at room temperature. Unbound material is then removed and wells are separated and counted via gamma scintillation.

Other Receptor Binding Assays

Whole cell assays for the determination of the binding affinity of pharmaceuticals of the present invention for the VEGF receptors, Flk-1/KDR and Flt-1, are described in Ortega, et. al., Amer. J. Pathol., 1997, 151, 1215-1224, and Dougher, et. al., Growth Factors, 1997, 14, 257-268. An in vitro assay for determining the affinity of pharmaceuticals of the present invention for the bFGF receptor is described in Yayon, et. al., Proc. Natl. Acad. Sci USA, 1993, 90, 10643-10647. Gho et. al., Cancer Research, 1997, 57, 3733-40, describe assays for angiogenin receptor binding peptides. Senger, et. al., Proc. Natl. Acad. Sci USA, 1997, 94, 13612-13617 describe assays for antagonists of the integrins a1B1 and a2B1. U.S. Pat. No. 5,536,814 describes assays for compounds that bind to the integrin a5B1.

Oncomouse® Imaging

The study involves the use of the c-Neu Oncomouse® and FVB mice simultaneously as controls. The mice are anesthetized with sodium pentobarbital and injected with approximately 0.5 mCi of radiopharmaceutical. Prior to injection, the tumor locations on each Oncomouse® are recorded and tumor size measured using calipers. The animals are positioned on the camera head so as to image the anterior or posterior of the animals. 5 Minute dynamic images are acquired serially over 2 hours using a 256×256 matrix and a zoom of 2×. Upon completion of the study, the images are evaluated by circumscribing the tumor as the target region of interest (ROI) and a background site in the neck area below the carotid salivary glands.

This model can also be used to assess the effectiveness of the radiopharmaceuticals of the present invention comprised of a beta, alpha or Auger electron emitting isotope. The radiopharmaceuticals are administered in appropriate amounts and the uptake in the tumors can be quantified either non-invasively by imaging for those isotopes with a coincident imageable gamma emission, or by excision of the tumors and counting the amount of radioactivity present by standard techniques. The therapeutic effect of the radiopharmaceuticals can be assessed by monitoring the rate of growth of the tumors in control mice versus those in the mice administered the radiopharmaceuticals of the present invention.

This model can also be used to assess the compounds of the present invention comprised of paramagnetic metals as MRI contrast agents. After administration of the appropriate amount of the paramagnetic compounds, the whole animal can be placed in a commercially available magnetic resonance imager to image the tumors. The effectiveness of the contrast agents can be readily seen by comparison to the images obtain from animals that are not administered a contrast agent.

This model can also be used to assess the compounds of the present invention comprised of heavy atoms as X-ray contrast agents. After administration of the appropriate amount of the X-ray absorbing compounds, the whole animal can be placed in a commercially available X-ray imager to image the tumors. The effectiveness of the contrast agents can be readily seen by comparison to the images obtain from animals that are not administered a contrast agent.

This model can also be used to assess the compounds of the present invention comprised of an echogenic gas containing surfactant microsphere as ultrasound contrast agents. After administration of the appropriate amount of the echogenic compounds, the tumors in the animal can be imaging using an ultrasound probe held proximate to the tumors. The effectiveness of the contrast agents can be readily seen by comparison to the images obtain from animals that are not administered a contrast agent.

Rabbit Matrigel Model

This model was adapted from a matrigel model intended for the study of angiogenesis in mice. Matrigel (Becton & Dickinson, USA) is a basement membrane rich in laminin, collagen IV, entactin, HSPG and other growth factors. When combined with growth factors such as bFGF [500 ng/ml] or VEGF [2 μg/ml] and injected subcutaneously into the mid-abdominal region of the mice, it solidifies into a gel and stimulates angiogenesis at the site of injection within 4-8 days. In the rabbit model, New Zealand White rabbits (2.5-3.0 kg) are injected with 2.0 ml of matrigel, plus 1 μg bFGF and 4 μg VEGF. The radiopharmaceutical is then injected 7 days later and the images obtained.

This model can also be used to assess the effectiveness of the radiopharmaceuticals of the present invention comprised of a beta, alpha or Auger electron emitting isotope. The radiopharmaceuticals are administered in appropriate amounts and the uptake at the angiogenic sites can be quantified either non-invasively by imaging for those isotopes with a coincident imageable gamma emission, or by excision of the angiogenic sites and counting the amount of radioactivity present by standard techniques. The therapeutic effect of the radiopharmaceuticals can be assessed by monitoring the rate of growth of the angiogenic sites in control rabbits versus those in the rabbits administered the radiopharmaceuticals of the present invention.

This model can also be used to assess the compounds of the present invention comprised of paramagnetic metals as MRI contrast agents. After administration of the appropriate amount of the paramagnetic compounds, the whole animal can be placed in a commercially available magnetic resonance imager to image the angiogenic sites. The effectiveness of the contrast agents can be readily seen by comparison to the images obtain from animals that are not administered a contrast agent.

This model can also be used to assess the compounds of the present invention comprised of heavy atoms as X-ray contrast agents. After administration of the appropriate amount of the X-ray absorbing compounds, the whole animal can be placed in a commercially available X-ray imager to image the angiogenic sites. The effectiveness of the contrast agents can be readily seen by comparison to the images obtain from animals that are not administered a contrast agent.

This model can also be used to assess the compounds of the present invention comprised of an echogenic gas containing surfactant microsphere as ultrasound contrast agents. After administration of the appropriate amount of the echogenic compounds, the angiogenic sites in the animal can be imaging using an ultrasound probe held proximate to the tumors. The effectiveness of the contrast agents can be readily seen by comparison to the images obtain from animals that are not administered a contrast agent.

Canine Spontaneous Tumor Model

Adult dogs with spontaneous mammary tumors were sedated with xylazine (20 mg/kg)/atropine (1 ml/kg). Upon sedation the animals were intubated using ketamine (5 mg/kg)/diazepam (0.25 mg/kg) for full anethesia. Chemical restraint was continued with ketamine (3 mg/kg)/xylazine (6 mg/kg) titrating as necessary. If required the animals were ventilated with room air via an endotrachial tube (12 strokes/min, 25 ml/kg) during the study. Peripheral veins were catheterized using 20 G I.V. catheters, one to serve as an infusion port for compound while the other for exfusion of blood samples. Heart rate and EKG were monitored using a cardiotachometer (Biotech, Grass Quincy, Mass.) triggered from a lead II electrocardiogram generated by limb leads. Blood samples are generally taken at ˜10 minutes (control), end of infusion, (1 minute), 15 min, 30 min, 60 min, 90 min, and 120 min for whole blood cell number and counting. Radiopharmaceutical dose was 300 μCi/kg administered as an i.v. bolus with saline flush. Parameters were monitored continuously on a polygraph recorder (Model 7E Grass) at a paper speed of 10 mm/min or 10 mm/sec.

Imaging of the laterals were for 2 hours with a 256×256 matrix, no zoom, 5 minute dynamic images. A known source is placed in the image field (20-90 μCi) to evaluate region of interest (ROI) uptake. Images were also acquired 24 hours post injection to determine retention of the compound in the tumor. The uptake is determined by taking the fraction of the total counts in an inscribed area for ROI/source and multiplying the known μCi. The result is μCi for the ROI.

This model can also be used to assess the effectiveness of the radiopharmaceuticals of the present invention comprised of a beta, alpha or Auger electron emitting isotope. The radiopharmaceuticals are administered in appropriate amounts and the uptake in the tumors can be quantified either non-invasively by imaging for those isotopes with a coincident imageable gamma emission, or by excision of the tumors and counting the amount of radioactivity present by standard techniques. The therapeutic effect of the radiopharmaceuticals can be assessed by monitoring the size of the tumors over time.

This model can also be used to assess the compounds of the present invention comprised of paramagnetic metals as MRI contrast agents. After administration of the appropriate amount of the paramagnetic compounds, the whole animal can be placed in a commercially available magnetic resonance imager to image the tumors. Effectiveness of the contrast agents can be readily seen by comparison to images obtained from animals that are not administered a contrast agent.

This model can also be used to assess the compounds of the present invention comprised of heavy atoms as X-ray contrast agents. After administration of the appropriate amount of the X-ray absorbing compounds, the whole animal can be placed in a commercially available X-ray imager to image the tumors. The effectiveness of the contrast agents can be readily seen by comparison to the images obtain from animals that are not administered a contrast agent.

Obviously, numerous modifications and variations of the present invention are possible in light of the above teachings. It is therefore to be understood that within the scope of the appended claims, the invention may be practiced otherwise that as specifically described herein.

1. A polypodal chelant having the formula: 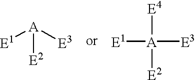
and pharmaceutically acceptable salts thereof, wherein A is a spacer selected from the group consisting of R¹—C, R¹—Si, R¹—Ge, N, P and P(O), or a macrocyclic group having the formula:
—[C(L)R²(CR³R⁴)ₐ]_{b}—,
—[N(L)C(W)(CR⁵R⁶)_{c}]_{d}—,
—[OC(W)C(L)R⁷(CR⁸R⁹)ₑ]_{f}—
or
—{[NR¹⁰C(W)C(L)R¹¹(CR¹²R¹³)_{g}]ₕ[NR¹⁴C(W)(CR¹⁵R¹⁶)ᵢ]ⱼ}—,
wherein
a is an integer selected from 1 to 3;
b is an integer selected from 3 to 5;
c is an integer selected from 1 to 3;
d is an integer selected from 3 or 4;
e is an integer selected from 1 to 3;
f is an integer selected from 3 or 4;
g is an integer selected from 1 to 3;
h is an integer selected from 3 or 4;
i is an integer selected from 1 to 3;
j is an integer selected from 0 to 3;
L is a direct bond to E¹, E², E³, and E⁴;
W is H₂ or O;
R¹, R², R³, R⁴, R⁵, R⁶, R⁷, R⁸, R⁹, R¹⁰, R¹¹, R¹², R¹³, R¹⁴, R¹⁵, and R¹⁶ are independently selected at each occurrence from the group consisting of H, C₁-C₆ alkyl, C₃-C₆ cycloalkyl, C₁-C₆ fluoroalkyl, C₁-C₆ alkenyl, C₃-C₆ cycloalkenyl, C₁-C₆ fluoroalkenyl, benzyl, fluorobenzyl, phenyl and fluorophenyl;
E¹, E², E³, and E⁴ are chelating arms each independently having the formula:
(CR¹⁷R¹⁸)ₖ—Z—X—(CR¹⁹R²⁰)ₗNR²¹R²²
wherein
k is an integer selected from 0 to 3, provided that when A is N or —[N(L)C(W)(CR⁵R⁶)_{c}]_{d}—, k is 1-3;
l is an integer selected from 1 to 3;
Z is selected from the group consisting of a bond, O, NH, NR¹NR¹, ONH and N(OR¹);
X is selected from the group consisting of C(O), S(O)₂ and P(O)(OR¹);
R¹⁷, R¹⁸, R¹⁹, R²⁰, R²¹ and R²² are independently selected from the group consisting of H, C₁-C₁₀ alkyl substituted with 0-5 R²³, C₁-C₁₀ fluoroalkyl substituted with 0-5 R²³, C₂-C₁₀ alkenyl substituted with 0-5 R²³, C₂-C₁₀ fluoroalkenyl substituted with 0-5 R²³, aryl substituted with 0-5 R²³, C₇-C₁₆ alkaryl wherein the aryl is substituted with 0-5 R²³ and fluoroaryl substituted with 0-5 R²³; or R¹⁷ and R¹⁸, R¹⁹ and R²⁰ or R²¹ and R²² may be taken together to form a C₃-C₁₀ cycloalkyl or C₃-C₁₀ cycloalkenyl optionally interrupted with C(O)NH, NH, NHC(O), NHC(O)NH, NHC(S)NH, O, S, S(O), S(O)₂, P(O)(OR²⁴), P(O)(OR²⁴)O or P(O)(NHR²⁴)O, or to form a ═CH—R^{22a} group, wherein R^{22a} is aryl substituted with 0-5 R²³, or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, C₁-C₆ fluoroalkenyl, benzyl, fluorobenzyl, phenyl, and fluorophenyl.

2. A polypodal chelant according to claim 1, characterized by having four chelating arms.

3. A polypodal chelant according to claim 1, characterized by being tripodal.

4. A tripodal chelant according to claim 3, wherein A is a spacer selected from the group consisting of R¹—C, N, P, P(O) , and —[N(L)C(W)(CR⁵R⁶)_{c}]_{d}—; R¹, R⁵, and R⁶ are selected from the group consisting of H, C₁-C₆ alkyl, C₃-C₆ cycloalkyl, C₁-C₆ alkenyl, C₃-C₆ cycloalkyl, benzyl, and phenyl; E¹, E², and E³ are chelating arms each independently having the formula:
(CH₂)ₖ—NHCOCH₂NR²¹R²²
R²¹ and R²² are independently selected at each occurrence from the group consisting of H, C₁-C₁₀ alkyl substituted with 0-2 R²³, C₂-C₁₀ alkenyl substituted with 0-2 R²³, aryl substituted with 0-2 R²³, and C₇-C₁₆ alkaryl, wherein the aryl is substituted with 0-2 R²³, or R²¹ and R²² may be taken together to form a ═CH—R^{22a} group, wherein R^{22a} is aryl substituted with 0-5 R²³, or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, benzyl and phenyl.

5. A tripodal chelant according to claim 4, wherein A is a spacer selected from the group consisting of N, P(O), and —[N(L)C(W)(CR⁵R⁶)_{c}]_{d}—; R⁵ and R⁶ are independently selected at each occurrence from the group consisting of H, C₁-C₆ alkyl, C₃-C₆ cycloalkyl, phenyl and benzyl; E¹, E², and E³ are chelating arms each independently having the formula:
(CH₂)ₖ—NHCOCH₂NR²¹R²²
wherein R²¹ and R²² are independently selected from the group consisting of C₁-C₁₀ alkyl substituted with 0-2 R²³, and aryl substituted with 0-2 R²³, or R²¹ and R²² may be taken together to form a ═CH—R^{22a} group, wherein R^{22a} is aryl substituted with 0-5 R²³, or heterocycle substituted by 0-5 R²³; R²³ is selected from the group consisting of OH, C₁-C₃ hydroxyalkyl, COOH, PO(OH)₂ and S(O)₂OH.

6. A tripodal chelant according to claim 5, wherein A is a spacer selected from the group consisting of N, and P(O); E¹, E² and E³ are chelating arms each independently having the formula:
(CH₂)ₖ—NHCOCH₂NR²¹R²²
wherein k is 2-3; R²¹ is independently selected from the group consisting of CH₃, CH₂COOH, and CH₂PO(OH)₂; and R²² is independently selected from the group consisting of CH₂COOH, and CH₂PO(OH)₂.

7. A tripodal chelant according to claim 6, wherein A is N or P(O); E¹, E², and E³ are (CH₂)ₖ—NHCOCH₂N(CH₂COOH)₂, and k is 2-3.

8. A tripodal chelant according to claim 7, wherein A is N; E¹, E² and E³ are (CH₂)ₖ—NHCOCH₂N (CH₂COOH)₂, and k is 2-3.

9. A tripodal chelant according to claim 7, wherein A is N; E¹, E² and E³ are (CH₂)ₖ—NHCOCH₂N(CH₃)(CH₂COOH), and k is 2-3.

10. A tripodal chelant according to claim 7, wherein A is N; E¹, E², and E³ are (CH₂)ₖ—NHCOCH₂N═CH—R^{22a}, k is 2-3, and R^{22a} is 2-hydroxyphenyl.

11. A tripodal chelant according to claim 7, wherein A is N; E¹, E², and E³ are (CH₂)ₖ—NHCOCH₂N═CH—R^{22a}, k is 2-3, and R^{22a} is 4-(2-methyl-3-hydroxy-5-hydroxymethyl)pyridyl.

12. A tripodal chelant according to claim 5, wherein A is —[N(L)—CH₂CH₂—]₃—; and E¹, E², and E³ are chelating arms each independently having the formula:
COCH₂NR²¹R²².

13. A tripodal chelant according to claim 12, wherein A is —[N(L)—CH₂CH₂—]₃—; E¹, E², and E³ are chelating arms each independently having the formula:
COCH₂NR²¹R²²
wherein R²¹ and R²² are independently selected from the group consisting of CH₂COOH, and CH₂PO(OH)₂.

14. A tripodal chelant according to claim 13, wherein A is —[N(L)—CH₂CH₂—]₃—; and E¹, E², and E³ are COCH₂N(CH₂COOH)₂.

15. A radiopharmaceutical compound comprising a polypodal chelant according to claim 1, chelated with a radionuclide selected from the group consisting of ^{52m}Mn, ⁵²Fe, ⁵⁵Co, ⁶⁴Cu, ⁶⁷Cu, ⁶⁷Ga, ⁶⁸Ga, ⁹⁰Y, ^{94m}Tc, ^{99m}Tc, ¹⁰⁵Rh, ¹⁰⁹Pd, ¹¹¹In, ^{117m}Sn, ¹⁴⁹Pr, ¹⁵³Sm, ¹⁵⁹Gd, ¹⁶⁶Ho, ¹⁶⁹Yb, ¹⁷⁷Lu, ¹⁸⁶Re, ¹⁸⁸Re, ²⁰³Pb, ²¹¹Pb, and ²¹²Bi.

16. The radiopharmaceutical compound according to claim 15, wherein said polypodal chelant is characterized by having four chelating arms.

17. The radiopharmaceutical compound according to claim 15, wherein said polypodal chelant is characterized by being tripodal.

18. The radiopharmaceutical compound according to claim 17, wherein A of said tripodal chelant is a spacer selected from the group consisting of R¹—C, N, P, P(O), and
—[N(L)C(W)(CR⁵R⁶)_{c}]_{d}—; R¹, R⁵, and R⁶ are selected from the group consisting of H, C₁-C₆ alkyl, C₃-C₆ cycloalkyl, C₁-C₆ alkenyl, C₃-C₆ cycloalkyl, benzyl, and phenyl; E¹, E², and E³ are chelating arms each independently having the formula:
(CH₂)ₖ—NHCOCH₂NR²¹R²²
R²¹ and R²² are independently selected at each occurrence from the group consisting of H, C₁-C₁₀ alkyl substituted with 0-2 R²³, C₂-C₁₀ alkenyl substituted with 0-2 R²³, aryl substituted with 0-2 R²³, and C₇-C₁₆ alkaryl, wherein the aryl is substituted with 0-2 R²³ or R²¹ and R²² may be taken together to form a ═CH—R^{22a} group, wherein R^{22a} is aryl substituted with 0-5 R²³, or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, benzyl and phenyl.

19. The radiopharmaceutical compound according to claim 18, wherein A is a spacer selected from the group consisting of N, P(O), and —[N(L)C(W)(CR⁵R⁶)_{c}]_{d}—; R⁵ and R⁶ are independently selected at each occurrence from the group consisting of H, C₁-C₆ alkyl, C₃-C₆ cycloalkyl, phenyl and benzyl; E¹, E², and E³ are chelating arms each independently having the formula:
(CH₂)ₖ—NHCOCH₂NR²¹R²²
wherein R²¹ and R²² are independently selected from the group consisting of C₁-C₁₀ alkyl substituted with 0-2 R²³, and aryl substituted with 0-2 R²³, or R²¹ and R²² may be taken together to form a ═CH—R^{22a} group, wherein R^{22a} is aryl substituted with 0-5 R²³, or heterocycle substituted by 0-5 R²³; R²³ is selected from the group consisting of OH, C₁-C₃ hydroxyalkyl, COOH, PO(OH)₂ and S(O)₂OH.

20. The radiopharmaceutical compound according to claim 19, wherein A is N or P(O); E¹, E² and E³ are chelating arms each independently having the formula:
(CH₂)ₖ—NHCOCH₂NR²¹R²²
wherein k is 2-3; R²¹ is independently selected from the group consisting of CH₃, CH₂COOH, and CH₂PO(OH)₂; and R²² is independently selected from the group consisting of CH₂COOH, and CH₂PO(OH)₂.

21. The radiopharmaceutical compound according to claim 20, wherein A is N or P(O); k is 2-3; and E¹, E² and E³ are
(CH₂)ₖ—NHCOCH₂N (CH₂COOH)₂.

22. The radiopharmaceutical compound according to claim 21, wherein A is N; E¹, E² and E³ are (CH₂)ₖ—NHCOCH₂N(CH₂COOH)₂, and k is 2-3.

23. The radiopharmaceutical compound according to claim 21, wherein A is N; k is 2-3; and E¹, E² and E³ are (CH₂)ₖ—NHCOCH₂N (CH₃)(CH₂COOH).

24. The radiopharmaceutical compound according to claim 21, wherein A is N; E¹, E² and E³ are (CH₂)ₖ—NHCOCH₂N═CH—R^{22a}, k is 2-3, and R^{22a} is 2-hydroxyphenyl.

25. The radiopharmaceutical compound according to claim 21, wherein A is N; E¹, E² and E³ are (CH₂)ₖ—NHCOCH₂N═CH—R^{22a}, k is 2-3, and R^{22a} is 4-(2-methyl-3-hydroxy-5-hydroxymethyl)pyridyl.

26. The radiopharmaceutical compound according to claim 19, wherein A is —[N(L)—CH₂CH₂—]₃—; and E¹, E² and E³ are chelating arms each independently having the formula:
COCH₂NR²¹R²².

27. An MRI contrast agent comprising a polypodal chelant according to claim 1, chelated with a paramagnetic metal ion of atomic number 21-29, 42-44 or 58-70.

28. The MRI contrast agent according to claim 27, wherein said polypodal chelant is characterized by having four chelating arms.

29. The MRI contrast agent according to claim 28, wherein said polypodal chelant is characterized by being tripodal.

30. The MRI contrast agent according to claim 29, wherein A of said tripodal chelant is a spacer selected from the group consisting of R₁—C, N, P, P(O), and —[N(L)C(W)(CR⁵R⁶)_{c}]_{d}—; R¹, R⁵, and R⁶ are selected from the group consisting of H, C₁-C₆ alkyl, C₃-C₆ cycloalkyl, C₁-C₆ alkenyl, C₃-C₆ cycloalkyl, benzyl, and phenyl; E¹, E², and E³ are chelating arms each independently having the formula:
(CH₂)ₖ—NHCOCH₂NR²¹R²²
R²¹ and R²² are independently selected at each occurrence from the group consisting of H, C₁-C₁₀ alkyl substituted with 0-2 R²³, C₂-C₁₀ alkenyl substituted with 0-2 R²³, aryl substituted with 0-2 R²³, and C₇-C₁₆ alkaryl, wherein the aryl is substituted with 0-2 R²³, or R²¹ and R²² may be taken together to form a ═CH—R^{22a} group, wherein R^{22a} is aryl substituted with 0-5 R²³, or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, benzyl and phenyl.

31. The MRI contrast agent according to claim 30, wherein A is a spacer selected from the group consisting of N, P(O), and —[N(L)C(W)(CR⁵R⁶)_{c}]_{d}—; R⁵ and R⁶ are independently selected at each occurrence from the group consisting of H, C₁-C₆ alkyl, C₃-C₆ cycloalkyl, phenyl and benzyl; E¹, E², and E³ are chelating arms each independently having the formula:
(CH₂)ₖ—NHCOCH₂NR²¹R²²
wherein R²¹ and R²² are independently selected from the group consisting of C₁-C₁₀ alkyl substituted with 0-2 R²³, and aryl substituted with 0-2 R²³, or R²¹ and R²² may be taken together to form a ═CH—R^{22a} group, wherein R^{22a} is aryl substituted with 0-5 R²³, or heterocycle substituted by 0-5 R²³; R²³ is selected from the group consisting of OH, C₁-C₃ hydroxyalkyl, COOH, PO(OH)₂ and S(O)₂OH.

32. The MRI contrast agent according to claim 31, wherein A is N or P(O); E¹, E² and E³ are chelating arms each independently having the formula:
(CH₂)ₖ—NHCOCH₂NR²¹R²²
wherein k is 2-3; R²¹ is independently selected from the group consisting of CH₃, CH₂COOH, and CH₂PO(OH)₂; and R²² is independently selected from the group consisting of CH₂COOH, and CH₂PO(OH)₂.

33. The MRI contrast agent according to claim 32, wherein A is N or P(O); k is 2-3; and E¹, E² and E³ are
(CH₂)ₖ—NHCOCH₂N (CH₂COOH)₂.

34. The MRI contrast agent according to claim 33, wherein A is N; E¹, E² and E³ are (CH₂)ₖ—NHCOCH₂N(CH₂COOH)₂, and k is 2-3.

35. The MRI contrast agent according to claim 33, wherein A is N; k is 2-3; and E¹, E² and E³ are
(CH₂)ₖ—NHCOCH₂N (CH₃)(CH₂COOH).

36. The MRI contrast agent according to claim 33, wherein A is N; E¹, E² and E³ are (CH₂)ₖ—NHCOCH₂N═CH—R^{22a}, k is 2-3, and R^{22a} is 2-hydroxyphenyl.

37. The MRI contrast agent according to claim 33, wherein A is N; E¹, E² and E³ are (CH₂)ₖ—NHCOCH₂N═CH—R^{22a}, k is 2-3, and R^{22a} is 4-(2-methyl-3-hydroxy-5-hydroxymethyl)pyridyl.

38. The MRI contrast agent according to claim 31, wherein A is —[N(L)—CH₂CH₂—]₃—; and E¹, E² and E³ are chelating arms each independently having the formula COCH₂NR²¹R²².

39. An X-ray or CT contrast agent comprising a polypodal chelant according to claim 1, chelated with a heavy metal ion of atomic number 21-31, 39-50, 56-80, 82, 83 or 90.

40. The X-ray or CT contrast agent according to claim 39, wherein said polypodal chelant is characterized by having four chelating arms.

41. The X-ray or CT contrast agent according to claim 40, wherein said polypodal chelant is characterized by being tripodal.

42. The X-ray or CT contrast agent according to claim 41, wherein A of said tripodal chelant is a spacer selected from the group consisting of R¹—C, N, P, P(O), and
—[N(L)C(W)(CR⁵R⁶)_{c}]_{d}—; R¹, R⁵, and R⁶ are selected from the group consisting of H, C₁-C₆ alkyl, C₃-C₆ cycloalkyl, C₁-C₆ alkenyl, C₃-C₆ cycloalkyl, benzyl, and phenyl; E¹, E², and E³ are chelating arms each independently having the formula:
(CH₂)ₖ—NHCOCH₂NR²¹R²²
R²¹ and R²² are independently selected at each occurrence from the group consisting of H, C₁-C₁₀ alkyl substituted with 0-2 R²³, C₂-C₁₀ alkenyl substituted with 0-2 R²³, aryl sub-stituted with 0-2 R²³, and C₇-C₁₆ alkaryl, wherein the aryl is substituted with 0-2 R²³, or R²¹ and R²² may be taken together to form a ═CH—R^{22a} group, wherein R^{22a} is aryl substituted with 0-5 R²³, or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, benzyl and phenyl.

43. The X-ray or CT contrast agent according to claim 42, wherein A is a spacer selected from the group consisting of N, P(O), and —[N(L)C(W)(CR⁵R⁶)_{c}]_{d}—; R⁵ and R⁶ are independently selected at each occurrence from the group consisting of H, C₁-C₆ alkyl, C₃-C₆ cycloalkyl, phenyl and benzyl; E¹, E², and E³ are chelating arms each independently having the formula:
(CH₂)ₖ—NHCOCH₂NR²¹R²²
wherein R²¹ and R²² are independently selected from the group consisting of C₁-C₁₀ alkyl substituted with 0-2 R²³, and aryl substituted with 0-2 R²³, or R²¹ and R²² may be taken together to form a ═CH—R^{22a} group, wherein R^{22a} is aryl substituted with 0-5 R²³, or heterocycle substituted by 0-5 R²³; R²³ is selected from the group consisting of OH, C₁-C₃ hydroxyalkyl, COOH, PO(OH)₂ and S(O)₂OH.

44. The X-ray or CT contrast agent according to claim 43, wherein A is N or P(O); E¹, E² and E³ are chelating arms each independently having the formula:
(CH₂)ₖ—NHCOCH₂NR²¹R²²
wherein k is 2-3; R²¹ is independently selected from the group consisting of CH₃, CH₂COOH, and CH₂PO(OH)₂; and R²² is independently selected from the group consisting of CH₂COOH, and CH₂PO(OH)₂.

45. The X-ray or CT contrast agent according to claim 44, wherein A is N or P(O); k is 2-3; and E¹, E² and E³ are
(CH₂)ₖ—NHCOCH₂N (CH₂COOH)₂.

46. The X-ray or CT contrast agent according to claim 45, wherein A is N; E¹, E² and E³ are (CH₂)ₖ—NHCOCH₂N(CH₂COOH)₂, and k is 2-3.

47. The X-ray or CT contrast agent according to claim 45, wherein A is N; k is 2-3; and E¹, E² and E³ are
(CH₂)ₖ—NHCOCH₂N(CH₃)(CH₂COOH).

48. The X-ray or CT contrast agent according to claim 45, wherein A is N; E¹, E² and E³ are (CH₂)ₖ—NHCOCH₂N═CH—R^{22a}, k is 2-3, and R^{22a} is 2-hydroxyphenyl.

49. The X-ray or CT contrast agent according to claim 45, wherein A is N; E¹, E² and E³ are (CH₂)ₖ—NHCOCH₂N═CH—R^{22a}, k is 2-3, and R^{22a} is 4-(2-methyl-3-hydroxy-5-hydroxy-methyl)pyridyl.

50. The X-ray or CT contrast agent according to claim 43, wherein A is —[N(L)—CH₂CH₂—]₃—; and E¹, E² and E³ are chelating arms each independently having the formula:
COCH₂NR²¹R²².

51. A conjugate of the formula:
BFC—Lₙ—BM,
and pharmaceutically acceptable salts thereof,
wherein BFC is a polypodal chelant according to claim 1, in which one of R¹ to R²⁴ includes a bond to Lₙ;
Lₙ is a linking group of formula:
L¹—[Y¹(CR²⁵R²⁶)_{f}(Z¹)_{f″}Y²]_{f′}—L²,
L¹ is —[(CH₂)_{g}Z¹]_{g′}—(CR²⁵R²⁶)_{g″}—;
L² is —(CR²⁵R²⁶)_{g″}—[Z¹(CH₂)_{g}]_{g′}—;
g is independently 0-10;
g′ is independently 0-1;
g″ is independently 0-10;
f is independently 0-10;
f′ is independently 0-10;
f″ is independently 0-1;
Y¹ and Y², at each occurrence, are independently selected from the group consisting of a bond, O, NR²⁶, C═O, C(═O)O, OC(═O)O, C(═O)NH—, C═NR²⁶, S, S(O), S(O)₂, NHC(═O), (NH)₂C(═O) and (NH)₂C═S;
R²⁵ and R²⁶ are independently selected at each occurrence from the group consisting of H, C₁-C₁₀ alkyl substituted with 0-5 R²⁷ and alkaryl wherein the aryl is substituted with 0-5 R²⁷;
R²⁷ is independently selected at each occurrence from the group consisting of NHR²⁸, C(═O)R²⁸, OC(═O)R²⁸, OC(═O)OR²⁸, C(═O)OR²⁸, C(═O)NR₂²⁸, —CN, SR²⁸, S(O)R²⁸, S(O)₂R²⁸, NHC(═O)R²⁸, NHC(═O)NHR²⁸, NHC(═S)NHR²⁸ and a bond to BM;
R²⁸ is independently selected at each occurrence from the group consisting of H, C₁-C₆ alkyl, benzyl, phenyl and a bond to BM; and
BM is a biologically active molecule selected from the group consisting of IIb/IIIa receptor ligands, fibrin binding peptides, leukocyte binding peptides, chemotactic peptides, LTB₄ receptor antagonists, somatostatin analogs, selectin binding peptides, vitronectin receptor antagonists, tyrosine kinase inhibitors, matrix metalloproteinase inhibitors, oligonucleotides, fatty acids, nitroimidazoles, and carbohydrates.

52. A conjugate according to claim 51, wherein said polypodal chelant is characterized by having four chelating arms.

53. A conjugate according to claim 51, wherein said polypodal chelant is characterized by being tripodal.

54. A conjugate according to claim 53, wherein A of said tripodal chelant is a spacer selected from the group consisting of R¹—C, N, P, P (O), and —[N(L)C(W)(CR⁵R⁶)_{c}]_{d}—; R¹, R⁵, and R⁶ are selected from the group consisting of H, C₁-C₆ alkyl, C₃-C₆ cycloalkyl, C₁-C₆ alkenyl, C₃-C₆ cycloalkyl, benzyl, and phenyl; E¹, E², and E³ are chelating arms each independently having the formula:
(CH₂)ₖ—NHCOCH₂NR²¹R²²
R²¹ and R²² are independently selected at each occurrence from the group consisting of H, C₁-C₁₀ alkyl substituted with 0-2 R²³, C₂-C₁₀ alkenyl substituted with 0-2 R²³, aryl sub-stituted with 0-2 R²³, and C₇-C₁₆ alkaryl, wherein the aryl is substituted with 0-2 R²³, or R²¹ and R²² may be taken together to form a ═CH—R^{22a} group, wherein R^{22a} is aryl substituted with 0-5 R²³, or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, benzyl and phenyl.

55. A conjugate according to claim 54, wherein A is a spacer selected from the group consisting of N, P(O) and —[N(L)C(W)(CR⁵R⁶)_{c}]_{d}—; R⁵ and R⁶ are independently selected at each occurrence from the group consisting of H, C₁-C₆ alkyl, C₃-C₆ cycloalkyl, phenyl and benzyl; E¹, E², and E³ are chelating arms each independently having the formula:
(CH₂)ₖ—NHCOCH₂NR²¹R²²
wherein R²¹ and R²² are independently selected from the group consisting of C₁-C₁₀ alkyl substituted with 0-2 R²³, and aryl substituted with 0-2 R²³, or R²¹ and R²² may be taken together to form a ═CH—R^{22a} group, wherein R^{22a} is aryl substituted with 0-5 R²³, or heterocycle substituted by 0-5 R²³; R²³ is selected from the group consisting of OH, C₁-C₃ hydroxyalkyl, COOH, PO(OH)₂ and S(O)₂OH.

56. A conjugate according to claim 55, wherein A is N or P(O); E¹, E² and E³ are chelating arms each independently having the formula:
(CH₂)ₖ—NHCOCH₂NR²¹R²²
wherein k is 2-3; R²¹ is independently selected from the group consisting of CH₃, CH₂COOH, and CH₂PO(OH)₂; and R²² is independently selected from the group consisting of CH₂COOH, and CH₂PO(OH)₂.

57. A conjugate according to claim 56, wherein A is N or P(O); E¹, E² and E³ are (CH₂)ₖ—NHCOCH₂N(CH₂COOH)₂, and k is 2-3.

58. A conjugate according to claim 57, wherein A is N; E¹, E², and E³ are (CH₂)ₖ—NHCOCH₂N(CH₂COOH)₂, and k is 2-3.

59. A conjugate according to claim 57, wherein A is N; E¹, E², and E³ are (CH₂)ₖ—NHCOCH₂N(CH₃)(CH₂COOH), and k is 2-3.

60. A conjugate according to claim 57, wherein A is N; E¹, E² and E³ are (CH₂)ₖ—NHCOCH₂N═CH—R^{22a}, k is 2-3, and R^{22a} is 2-hydroxyphenyl.

61. A conjugate according to claim 57, wherein A is N; E¹, E² and E³ are (CH₂)ₖ—NHCOCH₂N═CH—R^{22a}, k is 2-3, and R^{22a} is 4-(2-methyl-3-hydroxy-5-hydroxymethyl)pyridyl.

62. A conjugate according to claim 55, wherein A is —[N(L)—CH₂CH₂—]₃—; and E¹, E² and E³ are chelating arms each independently having the formula:
COCH₂NR²¹R²².

63. A radiopharmaceutical compound comprising a conjugate according to claim 51, chelated with a radionuclide selected from the group consisting of ^{52m}Mn, ⁵²Fe, ⁵⁵Co, ⁶⁴Cu, ⁶⁷Cu, ⁶⁷Ga, ⁶⁸Ga, ⁹⁰Y, ^{94m}Tc, ^{99m}Tc, ¹⁰⁵Rh, ¹⁰⁹Pd, ¹¹¹In, ^{117m}Sn, ¹⁴⁹Pr, ¹⁵³Sm, ¹⁵⁹Gd, ¹⁶⁶Ho, ¹⁶⁹Yb, ¹⁷⁷Lu, ¹⁸⁶Re, ¹⁸⁸Re, ²⁰³Pb, ²¹¹Pb, and ²¹²Bi.

64. An MRI contrast agent comprising a conjugate according to claim 51, chelated with a paramagnetic metal ion of atomic number 21-29, 42-44 or 58-70.

65. An X-ray or CT contrast agent comprising a conjugate according to claim 51, chelated with a heavy metal ion of atomic number 21-31, 39-50, 56-80, 82, 83 or 90.

66. A radiopharmaceutical composition for treating pathological processes involving angiogenic neovasculature in a patient in need thereof comprising a therapeutically effective amount of the radiopharmaceutical compound of claim 15 and a pharmaceutically acceptable carrier.

67. The composition of claim 66, wherein said radiopharmaceutical compound comprises a beta, alpha or Auger electron-emitting isotope.

68. A method for treating pathological processes involving angiogenic neovasculature in a patient in need thereof comprising administering to said patient a therapeutically effective amount of the radiopharmaceutical composition of claim 66.

69. A composition for radioactive imaging comprising an effective amount of the radiopharmaceutical compound of claim 15 and a pharmaceutically acceptable carrier.

70. A method for radioactive imaging comprising administering to a patient to be imaged sufficiently in advance thereto an effective amount of the radioactive imaging composition of claim 69.

71. A method according to claim 70, wherein said imaging method is gamma scintigraphy or positron-emission tomography.

72. A composition for X-ray imaging comprising an effective amount of the contrast agent of claim 39 and a pharmaceutically acceptable carrier.

73. A method for X-ray imaging comprising administering to a patient to be imaged sufficiently in advance thereof an effective amount of the X-ray imaging composition of claim 72.

74. A method according to claim 73, wherein said X-ray imaging method is CT imaging.

75. A composition for magnetic resonance imaging comprising an effective amount of the contrast agent of claim 27 and a pharmaceutically acceptable carrier.

76. A method for magnetic resonance imaging comprising administering to a patient to be imaged sufficiently in advance thereof an effective amount of the magnetic resonance imaging composition of claim 75.

77. A pharmaceutical composition for treating heavy metal toxicity in a patient in need thereof, comprising a therapeutically effective amount of the polypodal chelant of claim 1 and a pharmaceutically acceptable carrier.

78. A method for treating heavy metal toxicity in a patient in need thereof, comprising administering to said patient a therapeutically effective amount of the pharmaceutical composition of claim 77.

79. A radiopharmaceutical treatment kit comprising: a sterile, non-pyrogenic formulation comprising a radiopharmaceutical composition according to claim 66, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition. solubilization aids, bacteriostats and equipment for administering said composition.

80. The treatment kit of claim 79, wherein said formulation is in the form of a sterile solution or lyophilized solid.

81. A diagnostic kit comprising: a sterile, non-pyrogenic formulation comprising a radiopharmaceutical composition according to claim 66, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

82. The diagnostic kit of claim 81, wherein said formulation is in the form of a sterile solution or lyophilized solid.

83. A diagnostic kit comprising: a sterile, non-pyrogenic formulation comprising an X-ray imaging composition according to claim 72, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

84. The diagnostic kit of claim 83, wherein said formulation is in the form of a sterile solution or lyophilized solid.

85. A diagnostic kit comprising: a sterile, non-pyrogenic formulation comprising a magnetic resonance imaging composition according to claim 75, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

86. The diagnostic kit of claim 85, wherein said formulation is in the form of a sterile solution or lyophilized solid.

87. A compound having the formula:
A[(CR¹⁷R¹⁸)ₖNH₂]ₘ
wherein A is a spacer selected from the group consisting of R¹—C, R¹—Si, R¹—Ge, N, P and P(O), or a macrocyclic group having the formula:
—[C(L)R²(CR³R⁴)ₐ]_{b}—,
—[N(L)C(W)(CR⁵R⁶)_{c}]_{d}—,
—[OC(W)C(L)R⁷(CR⁸R⁹)ₑ]_{f}—
or
—{[NR¹⁰C(W)C(L)R¹¹(CR¹²R¹³)_{g}]ₕ[NR¹⁴C(W)(CR¹⁵R¹⁶)ᵢ]ⱼ}—,
wherein
a is an integer selected from 1 to 3;
b is an integer selected from 3 to 5;
c is an integer selected from 1 to 3;
d is an integer selected from 3 or 4;
e is an integer selected from 1 to 3;
f is an integer selected from 3 or 4;
g is an integer selected from 1 to 3;
h is an integer selected from 3 or 4;
i is an integer selected from 1 to 3;
j is an integer selected from 0 to 3;
k is an integer selected from 0 to 3;
m is an integer selected from 3 or 4;
L is a direct bond to [(CR¹⁷R¹⁸)ₖNH₂];
W is H₂ or O;
R¹, R², R³, R⁴, R⁵, R⁶, R⁷, R⁸, R⁹, R¹⁰, R¹¹, R¹², R¹³, R¹⁴, R¹⁵, and R¹⁶ are independently selected at each occurrence from the group consisting of H, C₁-C₆ alkyl, C₃-C₆ cycloalkyl, C₁-C₆ fluoroalkyl, C₁-C₆ alkenyl, C₃-C₆ cycloalkenyl, C₁-C₆ fluoroalkenyl, benzyl, fluorobenzyl, phenyl and fluorophenyl;
R¹⁷ and R¹⁸ are independently selected from the group consisting of H, C₁-C₁₀ alkyl substituted with 0-5 R²³,
C₁-C₁₀ fluoroalkyl substituted with 0-5 R²³, C₂-C₁₀ alkenyl substituted with 0-5 R²³, C₂-C₁₀ fluoroalkenyl substituted with 0-5 R²³, aryl substituted with 0-5 R²³, C₇-C₁₆ alkaryl wherein the aryl is substituted with 0-5 R²³, and fluoroaryl substituted with 0-5 R²³; or R¹⁷ and R¹⁸ may be taken together to form a C₃-C₁₀ cycloalkyl or C₃-C₁₀ cycloalkenyl optionally interrupted with C(O)NH, NH, NHC(O), NHC(O)NH, NHC(S)NH, O, S, S(O), S(O)₂, P(O)(OR²⁴), P(O)(OR²⁴)O or P(O)(NHR²⁴)O, or to form a ═CH—R^{22a} group, wherein R^{22a} is aryl substituted with 0-5 R²³ or heterocycle substituted by 0-5 R²³;
R²³ is selected from the group consisting of H, OH, C₁-C₃ alkyl, C₁-C₃ hydroxyalkyl, C(═O)R²⁴, C(═O)OR²⁴, C(═O)NR²⁴₂, PO(OR²⁴)₂ and S(O)₂OR²⁴; and
R²⁴ is selected from the group consisting of H, C₁-C₆ alkyl, C₃-C₆ cycloalkyl, C₁-C₆ fluoroalkyl, C₁-C₆ alkenyl, C₃-C₆ cycloalkyl, C₁-C₆ fluoroalkenyl, benzyl, fluorobenzyl, phenyl, and fluorophenyl.

88. A compound according to claim 60, wherein m is 4.

89. A compound according to claim 60, wherein m is 3.

90. A compound according to claim 62, wherein A is N or —[N(L)—C₂H₅]₃—; k is 0, 2 or 3; and R¹⁷ and R¹⁸ are H.

91. A compound according to claim 89, which is selected from the group consisting of:

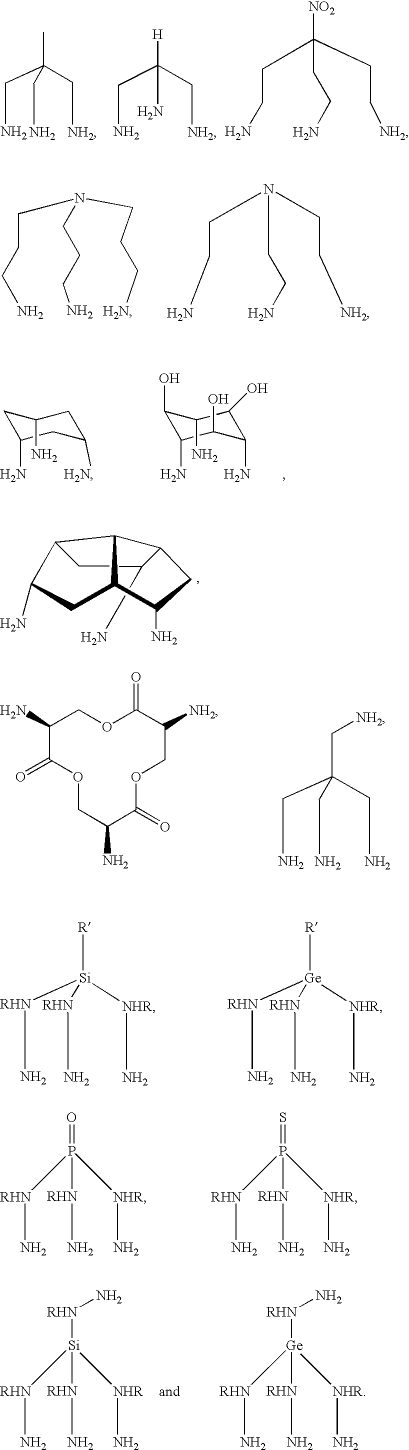

92. A radiopharmaceutical composition for treating pathological processes involving angiogenic neovasculature in a patient in need thereof comprising a therapeutically effective amount of the radiopharmaceutical compound of claim 63 and a pharmaceutically acceptable carrier.

93. The composition of claim 92, wherein said radiopharmaceutical compound comprises a beta, alpha or Auger electron-emitting isotope.

94. A method for treating pathological processes involving angiogenic neovasculature in a patient in need thereof comprising administering to said patient a therapeutically effective amount of the radiopharmaceutical composition of claim 92.

95. A composition for radioactive imaging comprising an effective amount of the radiopharmaceutical compound of claim 63 and a pharmaceutically acceptable carrier.

96. A method for radioactive imaging comprising administering to a patient to be imaged sufficiently in advance thereto an effective amount of the radioactive imaging composition of claim 95.

97. A method according to claim 96, wherein said imaging method is gamma scintigraphy or positron-emission tomography.

98. A composition for X-ray imaging comprising an effective amount of the contrast agent of claim 65 and a pharmaceutically acceptable carrier.

99. A method for X-ray imaging comprising administering to a patient to be imaged sufficiently in advance thereof an effective amount of the X-ray imaging composition of claim 98.

100. A method according to claim 99, wherein said X-ray imaging method is CT imaging.

101. A composition for magnetic resonance imaging comprising an effective amount of the contrast agent of claim 64 and a pharmaceutically acceptable carrier.

102. A method for magnetic resonance imaging comprising administering to a patient to be imaged sufficiently in advance thereof an effective amount of the magnetic resonance imaging composition of claim 101.

103. A radiopharmaceutical treatment kit comprising: a sterile, non-pyrogenic formulation comprising a radiopharmaceutical composition according to claim 92, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

104. The treatment kit of claim 103, wherein said formulation is in the form of a sterile solution or lyophilized solid.

105. A diagnostic kit comprising: a sterile, non-pyrogenic formulation comprising a radiopharmaceutical composition according to claim 92, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

106. The diagnostic kit of claim 105, wherein said formulation is in the form of a sterile solution or lyophilized solid.

107. A diagnostic kit comprising: a sterile, non-pyrogenic formulation comprising an X-ray imaging composition according to claim 98, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

108. The diagnostic kit of claim 107, wherein said formulation is in the form of a sterile solution or lyophilized solid.

109. A diagnostic kit comprising: a sterile, non-pyrogenic formulation comprising a magnetic resonance imaging composition according to claim 101, a pH 3-9 buffering agent and optionally one or more additives selected from the group consisting of ancillary ligands, reducing agents, transfer ligands, buffers, lyophilization aids, stabilization aids, solubilization aids, bacteriostats and equipment for administering said composition.

110. The diagnostic kit of claim 109, wherein said formulation is in the form of a sterile solution or lyophilized solid.
